SEQUENCE LISTING (17 sequences)

SEQ ID NO: 1 - protein, 178 aa, synthetic construct
  REGION 1..178: note = Human IL-10 Amino Acid Sequence
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 1629 bp, synthetic construct
  misc_feature 1..1629: note = Human IL-10 Nucleic Acid Sequence
  source 1..1629: mol_type = other DNA
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 3 - protein, 147 aa, synthetic construct
  REGION 1..147: note = EBV IL-10 Amino Acid Sequence
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 4 - DNA, 632 bp, synthetic construct
  misc_feature 1..632: note = EBV IL-10 Nucleic Acid Sequence
  source 1..632: mol_type = other DNA
tataaatcacttccctatctcaggtaggcctgcacaccttaggtatggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggaccctgaagccaaagaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccaggtgataattccataccctggaagcaggagatgggtgcatttcaccccaaccccccctttcgactgtcatttacaataaa

SEQ ID NO: 5 - protein, 147 aa, synthetic construct
  REGION 1..147: note = DV07 Amino Acid Sequence
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 6 - DNA, 441 bp, synthetic construct
  misc_feature 1..441: note = DV07 Nucleic Acid Sequence
  source 1..441: mol_type = other DNA
accgaccagtgcgacaacttccctcagatgctgcgggacctgagagatgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccaga

SEQ ID NO: 7 - protein, 713 aa, synthetic construct
  REGION 1..713: note = DK210egfr Amino Acid Sequence
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 8 - DNA, 2208 bp, synthetic construct
  misc_feature 1..2208: note = DK210egfr Nucleic Acid Sequence
  source 1..2208: mol_type = other DNA
gccgccaccatgggatggtctttgatcctgctgttcctggtggccgtggctaccagagtgcattctaccgaccagtgcgacaacttccctcagatgctgcgggacctgagggacgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccctgagatcaaggaccatgtgaactccctgggcgagaacctgaaaaccctgcggctgagactgcggcggtgtcacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggctagaggtggcggaggatctggcggtggtggttctggcggaggcggatctcaggttcagttgcaacaatggggcgctggcctgctgaagccttctgagacactgtctctgacctgcgccgtgtacggcttctctctgaccaattacggcgtgcactggatccggcagccacctggaaaaggactggaatggctgggagtgatttggagcggcggcaacaccgactacaacaccccttttacctctagagtggccatctccaaggacaactccaagaaccaggtgtccctgagactgaactctgtgaccgccgctgataccgccatctactactgtgctagagccctgacctactacgactacgagttcgcttattggggcaagggcaccaccgtgacagtttcatctggcggcggaggaagcggtggcggcggtagcggaggtggtggatctgctcctacctcctccagcaccaagaaaacccagctgcagttggagcatctgctgctggacctccagatgatcctgaacggcatcaacaactacaagaatcccaagctgacccggatgctgaccttcaagttctacatgcccaagaaggccaccgagctgaaacatctgcagtgcctggaagaggaactgaagcccctcgaggaagtgctgaatctggcccagtccaagaacttccacctgaggcctagggacctgatctccaacatcaacgtgatcgtgctcgagctgaagggctccgagacaacctttatgtgcgagtacgccgacgagacagccaccatcgtggaatttctgaaccggtggatcaccttctgccagtccatcatctctacattgaccggtggtggcggatcaggcggtggcggaagcggaggcggaggttctgaaattgtgctgacccagtctcctggcactctgtctttgagtcctggcgagagagctaccctgtcctgcagagcttctcagtccatcggcaccaacatccactggtatcagcagaagcctggacaggcccctcggctgctgattaagtacgcctctgagtccatcagcggcttccctgacagattctctggctctggatctggcaccgacttcaccctgaccatcaccagactggaacccgaggacttcgctatgtactactgccagcagaacaacaactggcccaccacctttggccagggcaccaagttggaaatcaaaggtggcggtggaagtggcggcggtggctcaggcggcggtggatctacagaccagtgtgataattttccacagatgctcagggatctccgcgacgcctttagccgggtcaagacattttttcagacaaaggatgaactcgataatctcctgctcaaagagagcctgctcgaggactttaaaggatacctgggatgccaggctctcagcgaaatgattcagttttatttggaggaagtcatgcctcaagctgaaaaccaggatccagagattaaggatcacgtcaacagcctcggcgagaatctcaagacactgcgcctgaggctgagaagatgccaccggtttctgccttgtgaaaacaagagcaaggctgtcgagcagattaagaatgcttttaacaaattgcaagaaaaagggatctataaggctatgtctgagtttgatatctttatcaattatatcgaagcttatatgactattaaggcccggtga

SEQ ID NO: 9 - protein, 733 aa, synthetic construct
  REGION 1..733: note = DK210her2 (Variant 4)
MGWSLILLFLVAVATRVHSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQEWGAGLLKPSETLSLTCAASGFNIKDTYIHWVRQPPGKGLEWVARIYPTNGYTRYADSVKGRFAISADTSKNQASLRLNSVTAADTAVYYCSRWGGDGFYAMDYWGKGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEIVMTQSPGTLSLSPGERATLSCRASQDVNTAVAWYQQKPGQAPRLLIYSASFLYSGVPDRFSGSRSGTDFTLTITRLEPEDFATYYCQQHYTTPPTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 10 - DNA, 2303 bp, synthetic construct
  misc_feature 1..2303: note = DK210her2 Variant 4 Nucleic Acid Sequence
  source 1..2303: mol_type = other DNA
atcgaaattaatacgactcactatagggagacccaagctggctagcgccgccaccatgggatggtctttgatcctgctgttcctggtggccgtggctaccagagtgcattctaccgaccagtgcgacaacttccctcagatgctgcgggacctgagagatgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccagaggcggcggaggatctggcggaggtggaagcggaggcggtggatctcaggtgcagttgcaagaatggggcgctggcctgctgaagccttccgaaacactgtctctgacctgcgccgccagcggcttcaacatcaaggacacctacatccactgggtccgacagcctccaggcaaaggactggaatgggtcgccagaatctaccccaccaacggctacaccagatacgccgactctgtgaagggcagattcgccatctctgccgacacctccaagaaccaggccagcctgagactgaactctgtgaccgctgctgacaccgccgtgtactactgctctagatggggcggagatggcttctacgccatggactattggggcaagggcaccaccgtgacagttagtagtggtggtggcggtagtggcggaggcggctcaggcggtggtggatctgctcctacatcctccagcaccaagaaaacccagctgcagttggagcatctgctgctggacctccagatgatcctgaacggcatcaacaactacaagaaccccaagctgacccggatgctgaccttcaagttctacatgcccaagaaggccaccgagctgaaacatctgcagtgcctggaagaggaactgaagcccctcgaggaagtgctgaatctggcccagtccaagaacttccacctgaggcctagggacctgatctccaacatcaacgtgatcgtgctcgagctgaagggctccgagacaaccttcatgtgcgagtacgccgacgagacagctaccatcgtggaatttctgaaccggtggatcaccttctgccagtccatcatctctaccctgactggtggcggaggaagcggcggaggcggatctggcggcggaggctctgaaattgtgatgacccagtctcctggcactctgtctctgtctcccggcgagagagctaccctgtcttgtagagccagccaggacgtgaacaccgctgtggcttggtatcagcagaagcctggacaggcccctcggctgctgatctactctgcctcctttctgtactccggcgtgcccgacagattctccggctctagatctggcaccgacttcaccctgaccatcaccagactggaacccgaggacttcgccacctactactgccagcagcactacaccacaccacctacctttggccagggcaccaagctggaaatcaaaggtggtggcggatcaggcggtggcggtagcggtggcggaggttctacagaccagtgtgataattttccccaaatgctgagggatctgcgggacgccttctctagggtcaagacattttttcagacaaaggatgaactcgataacctcttgctcaaagagagcctgctcgaggactttaagggatatctgggatgccaggctctgagcgaaatgattcagttttatttggaggaagtcatgcctcaagcagaaaaccaggatccagagattaaggatcatgtcaacagcctcggcgagaatctcaagacactgcgcctgaggctgagaagatgccaccggtttctgccttgtgaaaacaaaagcaaggctgtcgagcagattaagaatgcttttaacaaactccaagaaaaagggatctataaggctatgtctgagtttgatatctttatcaattatatcgaagcttatatgactattaaggctcgctaggggcccgtttaaacccgctgatcagcctcgactgtgccttctagtt

SEQ ID NO: 11 - protein, 733 aa, synthetic construct
  REGION 1..733: note = DK210her2 (Variant 5)
MGWSLILLFLVAVATRVHSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQEWGAGLLKPSETLSLTCAASGFNIKDTYIHWVRQPPGKGLEWVARIYPTNGYTRYADSVKGRFAISADTSKNQASLQMNSLRAEDTAVYYCSRWGGDGFYAMDYWGKGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEIQMTQSPSSLSLSPGERATLSCRASQDVNTAVAWYQQKPGQAPRLLIYSASFLYSGVPDRFSGSRSGTDFTLTISSLQPEDFATYYCQQHYTTPPTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 12 - DNA, 2303 bp, synthetic construct
  misc_feature 1..2303: note = DK210her2 Variant 5 Nucleic Acid Sequence
  source 1..2303: mol_type = other DNA
atcgaaattaatacgactcactatagggagacccaagctggctagcgccgccaccatgggatggtctttgatcctgctgttcctggtggccgtggctaccagagtgcattctaccgaccagtgcgacaacttccctcagatgctgcgggacctgagagatgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccagaggcggcggaggatctggcggaggtggaagcggaggcggtggatctcaggtgcagttgcaagaatggggcgctggcctgctgaagccttccgaaacactgtctctgacctgcgccgccagcggcttcaacatcaaggacacctacatccactgggtccgacagcctccaggcaaaggactggaatgggtcgccagaatctaccccaccaacggctacaccagatacgccgactctgtgaagggcagattcgccatctctgccgacacctccaagaaccaggccagcctgcagatgaacagcctgagagctgaggacaccgccgtgtactactgctctagatggggcggagatggcttctacgccatggactattggggcaagggcaccaccgtgacagttagtagtggtggtggcggtagtggcggaggcggctcaggcggtggtggatctgctcctacatcctccagcaccaagaaaacccagctgcagttggagcatctgctgctggacctccagatgatcctgaacggcatcaacaactacaagaaccccaagctgacccggatgctgaccttcaagttctacatgcccaagaaggccaccgagctgaaacatctgcagtgcctggaagaggaactgaagcccctcgaggaagtgctgaatctggcccagtccaagaacttccacctgaggcctagggacctgatctccaacatcaacgtgatcgtgctcgagctgaagggctccgagacaaccttcatgtgcgagtacgccgacgagacagctaccatcgtggaatttctgaaccggtggatcaccttctgccagtccatcatctctaccctgactggtggcggaggaagcggcggaggcggatctggcggcggaggctctgaaattcagatgacccagtctccttccagcctgtctctgtcccctggcgagagagctaccctgtcttgtagagccagccaggacgtgaacaccgctgtggcttggtatcagcagaagcctggacaggcccctcggctgctgatctactctgcctcctttctgtactccggcgtgcccgacagattctccggctctagatctggcaccgactttaccctgacaatcagctccctgcagcctgaggacttcgccacctactactgccagcagcactacaccacaccacctacctttggccagggcaccaagctggaaatcaaaggtggtggcggatcaggcggtggcggtagcggtggcggaggttctacagaccagtgtgataattttccccaaatgctgagggatctgcgggacgccttctctagggtcaagacattttttcagacaaaggatgaactcgataacctcttgctcaaagagagcctgctcgaggacttcaaaggatatctgggatgccaggctctgagcgaaatgattcagttttatttggaggaagtcatgcctcaagcagaaaaccaggatccagagattaaggatcatgtcaacagcctcggcgagaatctcaagacactgagactgaggctgcggagatgtcaccggtttctgccttgtgaaaacaagagcaaggctgtcgagcagattaagaatgcttttaacaaactccaagaaaaagggatctataaggctatgtctgagtttgatatctttatcaattatatcgaagcttatatgactattaaggctcgctaggggcccgtttaaacccgctgatcagcctcgactgtgccttctagtt

SEQ ID NO: 13 - protein, 732 aa, synthetic construct
  REGION 1..732: note = DK7DV07 EGFR
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 14 - protein, 721 aa, synthetic construct
  REGION 1..721: note = DK7DV07 HER2
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGSGGGSQVQLQQWGAGLLKPSETLSLTCAVYGFNIKDTYIHWIRQPPGKGLEWLGRIYPTNGYTRYADSVKGRVAISKDNSKNQVSLRLNSVTAADTAIYYCARWGGDGFYAMDYWGKGTTVTVSSGGGSGGGSGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGSGGGSGGGSEIVLTQSPGTLSLSPGERATLSCRASQDVNTAVAWYQQKPGQAPRLLIKSASFLYSGFPDRFSGSGSGTDFTLTITRLEPEDFAMYYCQQHYTTPPTFGQGTKLEIKGGGSGGGSGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 15 - protein, 722 aa, synthetic construct
  REGION 1..722: note = DK7DV07 CD20
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGSGGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSGGGSGGGSGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGSGGGSGGGSQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKGGGSGGGSGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 16 - protein, 735 aa, synthetic construct
  REGION 1..735: note = DK210 CD19
ATMGWSLILLFLVAVATRVHSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQEWGAGLLKPSETLSLTCTVSGVSLPDYGVSWIRQPPGKGLEWLGVIWGSETTYYNSALKSRVAISKDNSKNQVSLRLNSVTAADTAIYYCAKHYYYGGSYAMDYWGKGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEIVMTQSPGTLSLSPGERATLSCRASQDISKYLNWYQQKPGQAVRLLIYHTSRLHSGVPDRFSGSGSGTDYTLTITRLEPEDFATYFCQQGNTLPYTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 17 - protein, 735 aa, synthetic construct
  REGION 1..735: note = DK210 CD20
MGWSLILLFLVAVATRVHSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

(19) United States
(12) Patent Application Publication
(10) Pub. No.: US 2025/0026800 A1
(43) Pub. Date: Jan. 23, 2025

(54) METHOD OF REDUCING BISPECIFIC T CELL ENGAGER OR CHIMERIC ANTIGEN RECEPTOR T CELL MEDIATED CYTOKINE RELEASE SYNDROME USING INTERLEUKINS-4, -10, OR A FUSION PROTEIN THEREOF

(71) Applicant: Deka Biosciences, Inc., Germantown, MD (US)

(72) Inventor: John Mumm, Germantown, MD (US)

(21) Appl. No.: 18/829,057

(22) Filed: Sep. 9, 2024

Related U.S. Application Data
(63) Continuation of application No. 18/172,938, filed on Feb. 22, 2023.
(60) Provisional application No. 63/312,697, filed on Feb. 22, 2022.

(51) Int. Cl. C07K 14/54; A61K 35/17; A61P 35/00

(52) U.S. Cl. CPC C07K 14/5428; A61K 35/17; A61P 35/00; C07K 14/5406

(57) ABSTRACT

The disclosure provides for various methods including a method of reducing the severity of bispecific T cell engager (BiTE) or chimeric antigen receptor T cell (CAR-T) induced cytokine release syndrome (CRS) comprising administering to a patient in need thereof an amount of a composition comprising an interleukin 10 (IL-10) or an IL-10 agent, an interleukin 4 (IL-4) or an IL-4 agent, or combinations thereof.

### CROSS REFERENCE TO RELATED APPLICATIONS

This application is a Continuation of U.S. application Ser. No. 18/172,938 filed Feb. 23, 2023, which claims priority to U.S. Provisional Application No. 63/312,697, filed Feb. 22, 2022, the disclosures of which are incorporated by reference in their entirety.

### REFERENCE TO AN ELECTRONIC SEQUENCE LISTING

The contents of the electronic sequence listing (039451-00100-Sequence-Listing.xml; Size: 37,997 bytes; and Date of Creation: Feb. 22, 2023) are herein incorporated by reference in its entirety.

### BACKGROUND

Cytokine Release Syndrome (CRS) is a dose and treatment related toxicity resulting from Bispecific T cell Engager (BiTE) (Hosseini, 2020) activation of T cells and application of Chimeric Antigen Receptor T cells (CAR-T) to patients (Maude, 2014; Norelli, 2018). CRS is defined by the uncontrolled induction of high levels of interleukin 6 (IL-6) and interleukin 1 beta (IL-1β) predominantly (Liu, 2018), Tumor Necrosis Factor alpha (TNFα) (Chen, 2021) and interferon gamma (IFNγ) (Shimabukuro-Vornhagen, 2018) in the serum of treated patients.

To date, the molecular circuit responsible for the induction of CRS is not known. Based on the inventor's findings, it has been determined that the induction of CRS in BiTE and CAR-T patients is likely due to T Cell receptor (TCR):BiTE or CAR-T:Tumor Associated Antigen (TAA) clustering mediated activation of CD4+ T cells. This activation, in-turn, leads to the secretion of IL-2 (Brandl, 2007). The secretion of IL-2 then drives monocyte secretion of the proinflammatory cytokines associated with CRS (Bosco, 2000; Musso, 1992; Strieter, 1989).

### SUMMARY OF VARIOUS EMBODIMENTS

The inventor has found that treating monocytes with IL-10 or IL-4, IL-12, IL-15, IL-7 or any combination of the foregoing, or any half-life extended version thereof or any diakine comprising IL-10, IL-4, 11-12, IL-15, IL-7, or IL-2 that will engage cognate cytokine receptors on monocytes and directly inhibits the induction of proinflammatory cytokines by IL-2.

In one aspect, the application relates to a method of treating a monocyte with either IL-10 or IL-4, IL-12, IL-15, IL-7, half-life extended versions thereof, a combination of IL-10 and IL-4, IL-10 and IL-2, IL-10 and IL-7, IL-10 and IL-12, IL-10 and IL-15, or a fusion protein or diakine comprising at least two cytokines, wherein at least one of the at least two cytokines is IL-10 or IL-4, IL-12, 11-15, or IL-7 to reduce CRS associated with BiTE or CAR-T therapies. In one embodiment, monocytes are treated with IL-10, or IL-4, IL-12, IL-15, IL-7 or a half-life extended version thereof or a diakine comprising at least one of at least two cytokines is IL-10 or IL-4, IL-12, IL-15, or IL-7. In another embodiment a patient will be treated with IL-10 or a half-life extended version thereof and a BiTE or CAR-T. In yet another embodiment, a patient will be treated with IL-4 or a half-life extended version thereof and a BiTE or CAR-T. In another embodiment, a patient is treated with a diakine comprising IL-10 and any one of IFN-α, IL-2, IL-4, IL-7, IL-12, IL-15, IL-21 or IL-27 in an amount sufficient to reduce CRS. In yet another embodiment a patient is treated with a diakine comprising IL-4 and any one of IFN-α, IL-2, IL-7, IL-10, IL-12, IL-15, IL-21, or IL-27 in an amount sufficient to reduce CRS.

In another aspect, the application relates to a method of reducing BiTE or CAR-T associated CRS comprising administering to a subject thereof a BiTE or CAR-T therapeutic modality in combination with IL-10, IL-4, or any combination thereof, or a diakine comprising at least one of IL-10, IL-4, IL-2. In one embodiment, the method comprises administering to a patient in need thereof the BiTE or CAR-T therapy before, after or simultaneously with the IL-10 or IL-4, IL-12, IL-15, or IL-7, or half-life extended versions thereof, or a diakine comprising IL-10 or IL-4 combined with IFN-α, IL-2, IL-7, IL-12, IL-15, IL-21 or IL-27.

In yet another aspect, the application relates to a method of inhibiting the induction of proinflammatory cytokines in a patient undergoing BiTE or CAR-T therapy comprising administering to the patient undergoing said therapy a dose of IL-10 or IL-4 or half-life extended versions thereof, or a diakine comprising IL-10 or IL-4 in combination with IFN-α, IL-2, IL-7, IL-12, IL-15, IL-21 or IL-27 in an amount sufficient to suppress CRS caused by the proinflammatory cytokines.

The above simplified summary of representative aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

FIG. 1 are graphs measuring the level of IL1, IFNγ, TNFα, IFNα2a, IL-12, and IL-6 in PBMC in response to increasing levels of IL-2 exposure.

FIG. 2 are graphs measuring the levels of IFNγ, IL-6, and TNFα induction from PBMC to response to 1 μg/mL of anti-CD3 in the presence of a diakine, IL-10 and IL-2.

FIG. 3 mouse serum cytokines obtained from diakine exposure in vivo.

FIG. 4 non-human primate serum cytokine levels obtained from diakine exposure in vivo.

FIG. 5 graphs measuring cytotoxicity of CD8+ T cell exposed to diakine and BiTE.

FIG. 6 graphs measuring IFNg and TNFa levels in CD8+ T cells exposed to varying concentrations of BiTE.

FIG. 7 schematic diagram of proposed IL-2 mediated CRS circuit in response to BiTE or CAR-T therapy.

FIG. 8 combination of a diakine comprising IL-10 and IL-2 which targets EGFR in combination with CD3×CD19 BiTE enhances CD8+ T cell directed tumor cell cytolysis.

FIG. 9 combination of a diakine (DK2¹⁰ EGFR) with CD3×CD19 BiTE exhibits enhanced cytolytic effector molecules and controlled CRS

FIG. 10 intracellular FACS analysis of a diakine (DK2¹⁰ EGFR) and CD3×CD19 BiTE treatment of PBMC cultures containing Raji^{GFP+} tumor cells

### DETAILED DESCRIPTION

Exemplary aspects are described herein in the context of a using IL-10, IL-4, IL-12, IL-15, IL-7, or half-life extended versions thereof, or a diakine comprising at least one of IL-10 or IL-4 in combination with IFN-α, IL-2, IL-7, IL-12, IL-15, or IL-27 in a method of suppressing, inhibiting, reducing or preventing CRS associated with BiTE or CAR-T therapeutic modalities. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the exemplary aspects as illustrated in the accompanying drawings. The same reference indicators will be used to the extent possible throughout the drawings and the following description to refer to the same or like items.

Although a number of methods and materials similar or equivalent to those described herein can be used in the practice of the various described embodiments, the preferred materials and methods are described herein.

Unless otherwise indicated, the embodiments described herein employ conventional methods and techniques of molecular biology, biochemistry, pharmacology, chemistry, and immunology, which are well known to a person skilled in the art. See, e.g., Sambrook, et al., Molecular Cloning: A Laboratory Manual (2nd Edition, 1989); Methods In Enzymology (S. Colowick and N. Kaplan eds., Academic Press, Inc.); Handbook of Experimental Immunology, Vols. I-IV (D. M. Weir and C. C. Blackwell eds., Blackwell Scientific Publications); A. L. Lehninger, Biochemistry (Worth Publishers, Inc., current addition). Many of the general techniques for designing and fabricating diakines have been previously described in U.S. Application Publication No. 20220017587. This includes the types of IL-10 variants, including but not limited to human, mouse, CMV and/or EBV forms of IL-10, as well as the assays for testing the IL-10 variants, diakines, and other are known assay methods.

The following terms will be used to describe the various embodiments discussed herein, and are intended to be defined as indicated below.

As used herein in describing the various embodiments, the singular forms “a”, “an” and “the” include plural referents unless the content clearly dictates otherwise.

The term “about”, refers to a deviance of between 0.0001-5% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers. In a more specific embodiment, the term “about” refers to a difference of 1-25% in terms of nucleotide sequence homology or amino acid sequence homology when compared to a wild-type sequence.

The term “agent” as it relates to the various interleukins (IL), such “IL-10 agent” or “IL-4 agent” and the like, are intended to be construed broadly and include, for example, human and non-human forms of the interleukin polypeptides, including homologs, variants (including muteins), fragments thereof, and fusion proteins, as well as polypeptides having, for example, a leader sequence (e.g., the signal peptide), and modified versions of the foregoing. The present disclosure also contemplates nucleic acid molecules encoding the foregoing, vectors and the like containing the nucleic acid molecules, and cells (e.g., transformed cells and host cells) that express the interleukin agents. The terms “variant,” “analog” and “mutein” refer to biologically active derivatives of the reference molecule, that retain a desired activity, such as, for example, anti-inflammatory activity. Generally, the terms “variant,” “variants,” “analog” and “mutein” as it relates to a polypeptide refers to a compound or compounds having a native polypeptide sequence and structure with one or more amino acid additions, substitutions (which may be conservative in nature), and/or deletions, relative to the native molecule. For example, the terms “IL-10 variant”, “variant IL-10,” “IL-10 variant molecule,” and grammatical variations and plural forms thereof are all intended to be equivalent terms that refer to a variant forms of IL-10 amino acid (or nucleic acid) sequence that differs from wild-type IL-10 form anywhere from 1-25% in sequence identity or homology. Thus, for example, an EBV IL-10 variant molecule is one that differs from wild-type EBV IL-10 by having one or more amino acid (or nucleotide sequence encoding the amino acid) additions, substitutions and/or deletions.

The term “fusion protein” refers to a combination or conjugation of two or more proteins or polypeptides that results in a novel arrangement of proteins that do not normally exist naturally. The fusion protein is a result of covalent linkages of the two or more proteins or polypeptides. The two or more proteins that make up the fusion protein may be arranged in any configuration from amino-terminal end (“NH₂”) to carboxy-terminal end (“COOH”).

The term “homolog,” “homology,” “homologous” or “substantially homologous” refers to the percent identity between at least two polynucleotide sequences or at least two polypeptide sequences. Sequences are homologous to each other when the sequences exhibit at least about 50%, preferably at least about 75%, more preferably at least about 80%-85%, preferably at least about 90%, and most preferably at least about 95%-98% sequence identity over a defined length of the molecules.

The term “sequence identity” refers to an exact nucleotide-by-nucleotide or amino acid-by-amino acid correspondence. The sequence identity may range from 100% sequence identity to 50% sequence identity. A percent sequence identity can be determined using a variety of methods including but not limited to a direct comparison of the sequence information between two molecules (the reference sequence and a sequence with unknown percent identity to the reference sequence) by aligning the sequences, counting the exact number of matches between the two aligned sequences, dividing by the length of the reference sequence, and multiplying the result by 100. Readily available computer programs can be used to aid in the identification of percent identity.

The terms “subject,” “individual” or “patient” are used interchangeably herein and refer to a vertebrate, preferably a mammal. Mammals include, but are not limited to, murine, rodent, simian, human, farm animals, sport animals, and certain pets.

The term “administering” includes routes of administration which allow the active ingredient of the application to perform their intended function.

A “therapeutically effective amount” or “effective amount” as it relates to, for example, administering the IL variants, fusion proteins, dual cytokine fusion proteins, or diakine thereof described herein, refers to an amount sufficient to promote certain biological activities. These might include, for example, suppression of myeloid cell function, enhanced Kupffer cell activity, and/or lack of any effect on CD8′ T cells or enhanced CD8′ T-cell activity as well as blockade of mast cell upregulation of Fc receptor or prevention of degranulation or to promote or enhance to effects of combination therapeutics (e.g., CAR-T therapies) or to suppress the induction of cytokines from monocytes or macorphages. Thus, an “effective amount” will ameliorate or prevent a symptom or sign of the medical condition. Effective amount also means an amount sufficient to allow or facilitate diagnosis.

The term “treat,” “treating,” or “treatment” refers to a method of reducing the effects of a disease or condition. Treatment can also refer to a method of reducing the underlying cause of the disease or condition itself rather than just the symptoms. The treatment can be any reduction from native levels and can be, but is not limited to, the complete ablation of the disease, condition, or the symptoms of the disease or condition.

The term “half-life extended” as used in this application, refers to a protein that includes one of more additional moiety or moieties (such as proteins or PEGylation) that extends and/or enhances the circulation time within a subject anywhere in the range of 1-100 fold longer than a protein in the absence of the additional moiety or moieties. As it relates to IL, in one preferred embodiment, a half-life extended IL refers to an IL-10 or IL-4, IL-7, IL-12, or IL-15 conjugated to a scFv whereby the circulation time is extended in the range of 1-10 fold longer than the IL in the absence of the scFv. In another embodiment, a half-life extended version of an IL will adopt the configuration of formula I

NH₂-(Y)-(X¹)-(X²)-(Y)-COOH,

wherein
“Y” is any monomer from either a homodimeric or heterodimeric cytokine;
“X” is a VL or VH region obtained from a first monoclonal antibody;
“X²” is a VH or VL region obtained from the first monoclonal antibody,
wherein when X¹ is a VL, X² is a VH or when X¹ is a VH, X² is a VL and wherein the VH and VL together form a scFv.

The term “diakine” or “DK”, as used in this application, refers to a dual cytokine fusion protein comprising two monomers of a dimeric cytokine, which can be either a homodimer such as IL-10 or IL-10 variants, or a heterodimer such as IL-12 or IL-12 variants, that is fused together with a monomeric cytokine, such as IL-2, IL-4, IL-7, IL-15, IL-21, IL-28, IL-29, or with a dimeric cytokine, such as IL-12 or IL-10, whereby both of the dimeric cytokine and monomeric cytokine being fused onto a half-life extending antigen targeting domain. Representative diakines are described in detail in U.S. Pat. No. 11,292,822 (IL-10 based diakines) and co-pending U.S. application Ser. No. 18/065,504 (dual dimeric cytokine based diakines), both of which are incorporated by reference in their entireties. In one embodiment, a diakine, which may be used in a method in combination with a BiTE or CAR-T, is represented by formula II

NH₂-(Y)-(X¹)-(Zn)-(X²)-(Y)-COOH (Formula II)

wherein
“Y” is any monomer from either a homodimeric or heterodimeric cytokine;
“X” is a VL or VH region obtained from a first monoclonal antibody;
“X²” is a VH or VL region obtained from the first monoclonal antibody,
wherein when X′ is a VL, X² is a VH or when X′ is a VH, X² is a VL and wherein the VH and VL together form a scFv;
“Z” is a second cytokine, wherein the second cytokine is any monomeric cytokine or another dimeric cytokine; and
“n” is an integer selected from 0-2.

In one embodiment, dimeric cytokine may include IFN-α, IL-2, IL-4, IL-7, IL-10, IL-12, IL-15, IL-21, and IL-27. In another embodiments, the monomeric cytokine may include IL-6, IL-4, IL-1, IL-2, IL-3, IL-5, IL-7, IL-8, IL-9, IL-15, IL-21 IL-26, IL-27, IL-28, IL-29, GM-CSF, G-CSF, interferons-α, -β, -γ, TGF-β, or tumor necrosis factors-α, -β, basic FGF, EGF, PDGF, IL-4, IL-11, or IL-13. The VH and VL of the diakine is a scFv and may be derived from any monoclonal antibody but is preferably derived from an antibody that is capable of targeting a specific antigen. The monoclonal antibody from which the scFv (as it applies to Formula I and II) may be derived is selected from EGFR; CD52; CD14; various immune check point targets, such as but not limited to PD-L1, PD-1, TIM3, BTLA, LAG3 or CTLA4; CD19; CD20; CD22; CD47; GD-2; VEGFR1; VEGFR2; HER2; PDGFR; EpCAM; ICAM (ICAM-1, -2, -3, -4, -5), VCAM, CD14, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; CD123; CD33; BCMA; PSA; PSMA; CEA; GPC3; BCMA; DLL3; MUC17; CLDN 18; gpA33; HIV or Ebola. In an embodiment, the scFv (as it applies to Formula I and II) is an engrafted scFv, whereby the VH and VL framework region is derived from a first antibody (such as an anti-ebola antibody) and the CDRs are obtained from a second antibody (such as but not limited to EGFR; CD52; CD14; various immune check point targets, such as but not limited to PD-1, PD-1, TIM3, BTLA, LAG3 or CTLA4; CD19; CD20; CD22; CD47; GD-2; VEGFR1; VEGFR2; HER2; PDGFR; EpCAM; ICAM (ICAM-1, -2, -3, -4, -5), VCAM, CD14, FAPα; 5T4; Trop2; EDB-FN; TGFβ Trap; MAdCAM, P7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; CD123; CD33; BCMA; PSA; PSMA; CEA; GPC3; BCMA; DLL3; MUC17; CLDN 18; gpA33; HIV or Ebola). In one preferred embodiment, the scFv is derived from an anti-Ebola antibody where the VH and VL framework regions of an anti-ebola antibody are substituted or engrafted with 6 CDR regions from an antibody specific for EGFR; CD52; CD14; various immune check point targets, such as but not limited to PD-L1, PD-1, TIM3, BTLA, LAG3 or CTLA4; CD19; CD20; CD22; CD47; GD-2; VEGFR1; VEGFR2; HER2; PDGFR; EpCAM; ICAM (ICAM-1, -2, -3, -4, -5), VCAM, CD14, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; CD123; CD33; BCMA; PSA; PSMA; CEA; GPC3; BCMA; DLL3; MUC17; CLDN 18; gpA33; more preferably an anti-EGFR, anti-MAdCAM, anti-VEGFR1, anti-VEGFR2, anti-PDGFR, or anti-CD14, anti-CD19, anti-CD20, or anti-CD22.

In another embodiment, the diakine, which may be used in a method in combination with a BiTE or a CAR-T, is a diakine described in U.S. Pat. No. 11,292,822 (IL-10 based diakines) or co-pending U.S. application Ser. No. 18/065,504 (dual dimeric cytokine based diakines), both of which are incorporated by reference in their entireties. In yet another embodiment, the diakine, which may be used in a method in combination with a BiTE or a CAR-T, is protein represented by formula III

NH₂-(IL-10)-(X¹)-(Zn)-(X²)-(IL-10)-COOH (Formula III);

wherein
“IL-10” is a monomer;
“X” is a VL or VH region from a first monoclonal antibody;
“X²” is a VH or VL region from the first monoclonal antibody;
wherein when X′ is a VL, X² is a VH or when X′ is a VH, X² is a VL;
wherein the first monoclonal antibody is an anti-ebola antibody or the scFv framework region obtained therefrom;
wherein the VL and VH from the anti-ebola antibody include 3 light chain CDRs and 3 heavy chain CDRs that are engrafted with 3 light chain CDRs and 3 heavy chain CDRs from a second monoclonal antibody;
“Z” is a cytokine other than IL-10;
“n” is an integer of 1; and
   wherein the following variables in Table 1 apply to formula III:

TABLE 1

| | “second monoclonal | |
|---|---|---|
| “IL-10” monomer | antibody” | “Z” |
| Human IL-10 (Seq ID No: 1) | EGFR | 2 |
| | | 15 |
| | | 7 |
| | | 28 |
| | | 29 |
| | | IFN-alpha |
| | | 21 |
| | HER2 | 2 |
| | | 15 |
| | | 7 |
| | | 28 |
| | | 29 |
| | | IFN-alpha |
| | | 21 |
| | VEGFR2 | 2 |
| | | 15 |
| | | 7 |
| | | 28 |
| | | 29 |
| | | IFN-alpha |
| | | 21 |
| | PDGFR | 2 |
| | | 15 |
| | | 7 |
| | | 28 |
| | | 29 |
| | | IFN-alpha |
| | | 21 |
| | GPC3 | 2 |
| | | 15 |
| | | 7 |
| | | 28 |
| | | 29 |
| | | IFN-alpha |
| | | 21 |
| | PD-L1 | 2 |
| | | 15 |
| | | 7 |
| | | 28 |
| | | 29 |
| | | IFN-alpha |
| | | 21 |
| | CD19 | 2 |
| | | 15 |
| | | 7 |
| | | 28 |
| | | 29 |
| | | IFN-alpha |
| | | 21 |
| | CD20 | 2 |
| | | 15 |
| | | 7 |
| | | 28 |
| | | 29 |
| | | IFN-alpha |
| | | 21 |
| | CD22 | 2 |
| | | 15 |
| | | 7 |
| | | 28 |
| | | 29 |
| | | IFN-alpha |
| | | 21 |
| | PSMA | 2 |
| | | 15 |
| | | 7 |
| | | 28 |
| | | 29 |
| | | IFN-alpha |
| | | 21 |
| | CEA | 2 |
| | | 15 |
| | | 7 |
| | | 28 |
| | | 29 |
| | | IFN-alpha |
| | | 21 |
| | BCMA | 2 |
| | | 15 |
| | | 7 |
| | | 28 |
| | | 29 |
| | | IFN-alpha |
| | | 21 |
| | gpA33 | 2 |
| | | 15 |
| | | 7 |
| | | 28 |
| | | 29 |
| | | IFN-alpha |
| | | 21 |
| | CD33 | 2 |
| | | 15 |
| | | 7 |
| | | 28 |
| | | 29 |
| | | IFN-alpha |
| | | 21 |
| | DLL3 | 2 |
| | | 15 |
| | | 7 |
| | | 28 |
| | | 29 |
| | | IFN-alpha |
| | | 21 |
| | MUC17 | 2 |
| | | 15 |
| | | 7 |
| | | 28 |
| | | 29 |
| | | IFN-alpha |
| | | 21 |
| | CLDN18 | 2 |
| | | 15 |
| | | 7 |
| | | 28 |
| | | 29 |
| | | IFN-alpha |
| | | 21 |
| | GD2 | 2 |
| | | 15 |
| | | 7 |
| | | 28 |
| | | 29 |
| | | IFN-alpha |
| | | 21 |
| | 5T4 | 2 |
| | | 15 |
| | | 7 |
| | | 28 |
| | | 29 |
| | | IFN-alpha |
| | | 21 |
| | HER3 | 2 |
| | | 15 |
| | | 7 |
| | | 28 |
| | | 29 |
| | | IFN-alpha |
| | | 21 |
| EBV IL-10 (Seq ID No: 5) | EGFR | 2 |
| | | 15 |
| | | 7 |
| | | 28 |
| | | 29 |
| | | IFN-alpha |
| | | 21 |
| | HER2 | 2 |
| | | 15 |
| | | 7 |
| | | 28 |
| | | 29 |
| | | IFN-alpha |
| | | 21 |
| | VEGFR2 | 2 |
| | | 15 |
| | | 7 |
| | | 28 |
| | | 29 |
| | | IFN-alpha |
| | | 21 |
| | PDGFR | 2 |
| | | 15 |
| | | 7 |
| | | 28 |
| | | 29 |
| | | IFN-alpha |
| | | 21 |
| | GPC3 | 2 |
| | | 15 |
| | | 7 |
| | | 28 |
| | | 29 |
| | | IFN-alpha |
| | | 21 |
| | PD-L1 | 2 |
| | | 15 |
| | | 7 |
| | | 28 |
| | | 29 |
| | | IFN-alpha |
| | | 21 |
| | CD19 | 2 |
| | | 15 |
| | | 7 |
| | | 28 |
| | | 29 |
| | | IFN-alpha |
| | | 21 |
| | CD20 | 2 |
| | | 15 |
| | | 7 |
| | | 28 |
| | | 29 |
| | | IFN-alpha |
| | | 21 |
| | CD22 | 2 |
| | | 15 |
| | | 7 |
| | | 28 |
| | | 29 |
| | | IFN-alpha |
| | | 21 |
| | PSMA | 2 |
| | | 15 |
| | | 7 |
| | | 28 |
| | | 29 |
| | | IFN-alpha |
| | | 21 |
| | CEA | 2 |
| | | 15 |
| | | 7 |
| | | 28 |
| | | 29 |
| | | IFN-alpha |
| | | 21 |
| | BCMA | 2 |
| | | 15 |
| | | 7 |
| | | 28 |
| | | 29 |
| | | IFN-alpha |
| | | 21 |
| | gpA33 | 2 |
| | | 15 |
| | | 7 |
| | | 28 |
| | | 29 |
| | | IFN-alpha |
| | | 21 |
| | CD33 | 2 |
| | | 15 |
| | | 7 |
| | | 28 |
| | | 29 |
| | | IFN-alpha |
| | | 21 |
| | DLL3 | 2 |
| | | 15 |
| | | 7 |
| | | 28 |
| | | 29 |
| | | IFN-alpha |
| | | 21 |
| | MUC17 | 2 |
| | | 15 |
| | | 7 |
| | | 28 |
| | | 29 |
| | | IFN-alpha |
| | | 21 |
| | CLDN18 | 2 |
| | | 15 |
| | | 7 |
| | | 28 |
| | | 29 |
| | | IFN-alpha |
| | | 21 |
| | GD2 | 2 |
| | | 15 |
| | | 7 |
| | | 28 |
| | | 29 |
| | | IFN-alpha |
| | | 21 |
| | 5T4 | 2 |
| | | 15 |
| | | 7 |
| | | 28 |
| | | 29 |
| | | IFN-alpha |
| | | 21 |
| | HER3 | 2 |
| | | 15 |
| | | 7 |
| | | 28 |
| | | 29 |
| | | IFN-alpha |
| | | 21 |

In another embodiment, the diakine, which may be used in a method in combination with a BiTE or CAR-T, is a diakine described in co-pending U.S. application Ser. No. 18/065,504 in Tables 2a-2d and 3a-3d, which is incorporated by reference in its entirety.

In another aspect, the protein or nucleic acid molecules encoding dual cytokine fusion protein or DK may be formulated as a pharmaceutical composition comprising a therapeutically effective amount of the dual cytokine fusion protein and a pharmaceutical carrier and/or pharmaceutically acceptable excipients. The pharmaceutical composition may be formulated with commonly used buffers, excipients, preservatives, stabilizers. The pharmaceutical compositions comprising the dual cytokine fusion protein is mixed with a pharmaceutically acceptable carrier or excipient. Various pharmaceutical carriers are known in the art and may be used in the pharmaceutical composition. For example, the carrier can be any compatible, non-toxic substance suitable for delivering the dual cytokine fusion protein compositions of the application to a patient. Examples of suitable carriers include normal saline, Ringer's solution, dextrose solution, and Hank's solution. Carriers may also include any poloxamers generally known to those of skill in the art, including, but not limited to, those having molecular weights of 2900 (L64), 3400 (P65), 4200 (P84), 4600 (P85), 11,400 (F88), 4950 (P103), 5900 (P104), 6500 (P105), 14,600 (F108), 5750 (P123), and 12,600 (F127). Carriers may also include emulsifiers, including, but not limited to, polysorbate 20, polysorbate 40, polysorbate 60, and polysorbate 80, to name a few. Non-aqueous carriers such as fixed oils and ethyl oleate may also be used. The carrier may also include additives such as substances that enhance isotonicity and chemical stability, e.g., buffers and preservatives, see, e.g., Remington's Pharmaceutical Sciences and U.S. Pharmacopeia: National Formulary, Mack Publishing Company, Easton, Pa. (1984). Formulations of therapeutic and diagnostic agents may be prepared by mixing with physiologically acceptable carriers, excipients, or stabilizers in the form of lyophilized powders, slurries, aqueous solutions or suspensions, for example.

The pharmaceutical composition will be formulated for administration to a patient in a therapeutically effective amount sufficient to provide the desired therapeutic result. Preferably, such amount has minimal negative side effects. In one embodiment, the amount of dual cytokine fusion protein administered will be sufficient to treat or prevent inflammatory diseases or condition. In another embodiment, the amount of dual cytokine fusion protein administered will be sufficient to treat or prevent immune diseases or disorders. In still another embodiment, the amount of diakine or dual cytokine fusion protein administered will be sufficient to treat or prevent CRS mediated by BiTE or CAR-T therapy. The amount administered may vary from patient to patient and will need to be determined by considering the subject's or patient's disease or condition, the overall health of the patient, method of administration, the severity of side-effects, and the like.

An effective amount for a particular patient may vary depending on factors such as the condition being treated, the overall health of the patient, the method route and dose of administration and the severity of side effects. The appropriate dose administered to a patient is typically determined by a clinician using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

The method for determining the dosing of the presently described dual cytokine fusion protein will be substantially similar to that described in U.S. Pat. No. 10,858,412. Generally, the presently described dual cytokine fusion protein will have a dosing in the range of 0.01 mg/kg to 1 mg/kg, preferably 0.025 mg/kg to 0.5 mg/kg. The dual cytokine fusion protein may be administered daily, three times a week, twice a week, weekly, bimonthly, or monthly. An effective amount of therapeutic will impact the level of CRS inhibited caused by the BiTE or CAR-T therapy. In yet another embodiment, the diakine will be dosed at a concentration of 0.1 ng/mL to 200 ng/mL, preferably, 10 ng/mL to 100 ng/mL. Generally, the addition of a diakine will lower the required dose for a BiTE or CAR-T modality.

Compositions of the application can be administered orally or injected into the body. Formulations for oral use can also include compounds to further protect the IL or DK molecules from proteases in the gastrointestinal tract. Injections are usually intramuscular, subcutaneous, intradermal or intravenous. Alternatively, intra-articular injection or other routes could be used in appropriate circumstances. Parenterally administered dual cytokine fusion protein are preferably formulated in a unit dosage injectable form (solution, suspension, emulsion) in association with a pharmaceutical carrier and/or pharmaceutically acceptable excipients. In other embodiments, compositions of the application may be introduced into a patient's body by implantable or injectable drug delivery system.

Those of skill in the art will recognize that adoptive cell therapy (such as adoptive T-cell therapy) is well known and practiced according to procedures previously described. See, e.g., U.S. Pat. No. 4,690,915. These methods may include autologous transfer (i.e., derived from the patient) or allogenic transfer (i.e. derived from another subject other than the patient to be treated).

The CAR-T or TCR-T cells are administered by methods known and conventionally practiced by those familiar with adaptive cell therapy. In one embodiment, the administration method includes, but is not limited to bolus infusion, intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, or intralesional or intrtumoral administration. Parenteral infusions include intramuscular, intravenous, intraarterial, intraperitoneal, or subcutaneous administration. In certain some embodiments, the recombinantly engineered CAR-T or TCR-T is administered as a single bolus administration, multiple bolus or continuous infusion. Generally known CAR-T therapies include idecabtagene vicleucel, lisocabtagene maraleucel, brexucabtagene autoleucel, tisagenlecleucel, or axicabtagene ciloleucel.

In one embodiment, the diakine and the CAR-T are administered in separate subsequent time periods, wherein, for example, the diakine (such as DK2¹⁰vegfr2 or DK2¹⁰EGFR or any of those referenced in Table 1, above) is administered prior to the administration of a recombinantly engineered CAR-T cell.

In other embodiments, the diakine and the CAR-T are administered simultaneously. In other embodiments, the diakine is administered 1-3 days before the CAR-T therapy and then simultaneously administered along with the CAR-T, and/or 1-7 days following CAR-T administration. The diakine may be administered once a day or week, or 2-3 times a week in combination or conjunction with the CAR-T. In another aspect, the diakine is utilized in the expansion and/or thawing procedure of the CAR-T cells prior to administration. Upon reconstituting CAR-T cells from cryopreserved stock, the CAR-T are typically rested in the presence of CAR-T beneficial cytokines (e.g., low dose IL-2). In one aspect, the CAR-T cells may be primed or expanded from cryopreserved stocks in the presence of a diakine. In one aspect, the CAR-T is expanded or primed in the presence of 0.001 to 300 ng/mL of a diakine, more preferably 0.01 to 200 ng/mL of a diakine.

Likewise, the diakine and the BiTE are administered in separate subsequent time periods, wherein the diakine (e.g., DK2¹⁰CD20 or DK2¹⁰EGFR or DK2¹⁰HER2 or DK2¹⁰HER3) is administered 1-3 days before administering the BiTE (e.g., CD3×CD19 BiTE). In other embodiments, the diakine is administered 1-3 days before the BiTE and then simultaneously administered along with the BiTE, and/or 1-7 days following BiTE administration. The diakine may be administered once a day or week, or 2-3 times a week in combination or conjunction with the BiTE. A BiTE will generally follow the format of a bispecific antibody having an anti-CD3 and an anti-TAA fused together. In one embodiment, method combines an IL-10 or IL-4, or half-life extended versions thereof, or a diakine comprising IL-10 or IL-4 and IFN-α, IL-2, IL-7, IL-12, IL-15, IL-21 or IL-27, in conjunction with a BiTE, such as those that have bispecificity for CD3 with CD33, BCMA, CD19, CD20, CD22, PSMA, EGFR, DLL3, MUC17, CLDN18, CEA, HER2, HER3, EpCAM, gpA33, GPC3, GD2 5T4, VEGFR2, PDGFR, PDL1, or PD1 to name a few. In a preferred embodiment, the BiTE is an anti-CD3 and anti-CD19, anti-CD20, anti-HER2, anti-HER3, anti-PSMA, or anti-BCMA.

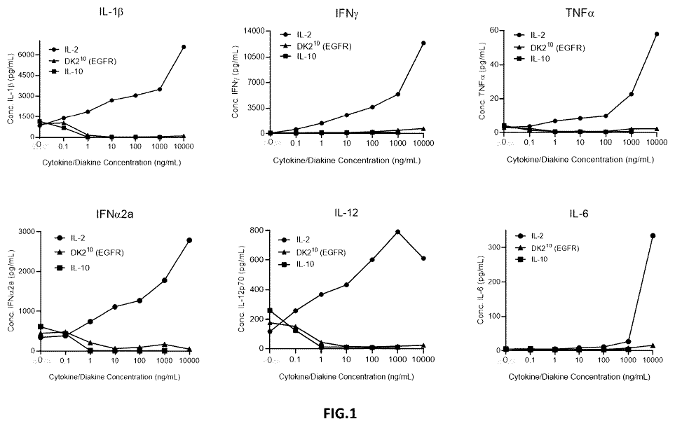

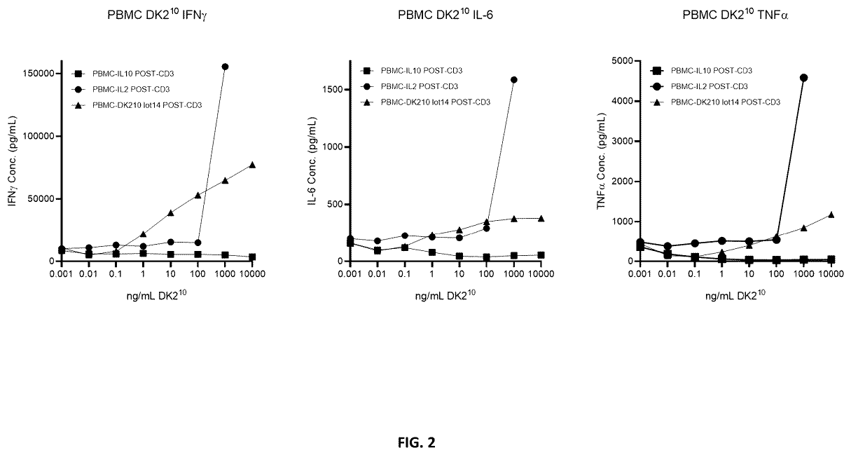

It is generally understood that CRS is associated by concomitant increases in serum IL-6, IL-1β, TNFα, IFNα/γ, IL-12 and IL-23. While these cytokines are generally associated with CRS, it is not known whether one, two or all of these cytokines working together to lead to the toxic side effects associated with CRS. The inventor believes that a potential root cause of CRS is the fact than many patients with severe CRS induced by BiTE or CAR-T treatment develop vascular leak syndrome. Vascular or capillary leak syndrome is also predominantly observed as the dose limiting and often lethal toxicity associated with high dose IL-2 therapy. In addition, the inventor has found that that treatment of human peripheral blood mononuclear cells (PBMC) or human whole blood cells with an increasing concentration of IL-2 leads to the induction of a panel of proinflammatory cytokines reminiscent of CRS. FIG. 1.

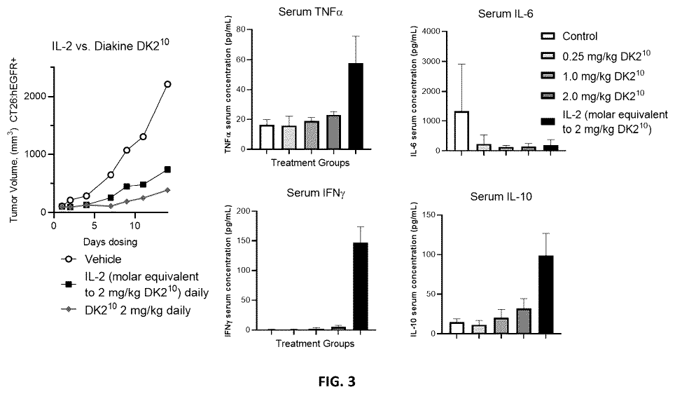

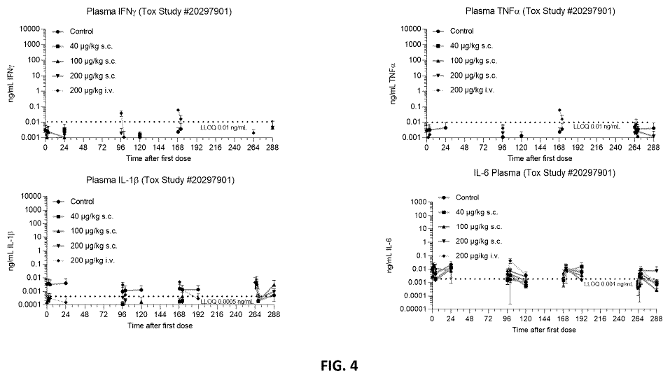

Without being bound to any particular theory, the inventor believes that the similarities between the cytokines induced by treatment with IL-2 alone, and the apparent blockade of the induction of these secondary cytokines by a diakine comprising IL-10 (e.g., DK2¹⁰ (EGFR)) or IL-4, IL-12, IL-15, IL-7, or any combination thereof will suppress or block IL-2 mediated CRS. Others have shown that the addition of tumor cells expressing specific tumor associated antigens (TAA) to PBMC with a titration range of an anti-TAA:anti-CD3 BiTE leads to the induction of a range of proinflammatory cytokines. (Fu, 2019). It also appears that BiTE stimulation temporally leads to the initial induction of TNFα, IL-2 and IL-4, followed by the other cytokines. (Brandl, 2007). The level of IFNγ, IL-6, and TNFα induction from PBMCs in response to exposure to anti-CD3 in the presence of a diakine (DK2¹⁰ EGFR), IL-10, and IL-2, suggest that the presence of DK2¹⁰ (EGFR) prevents the induction of significant secondary proinflammatory cytokines associated with CRS, unlike the increasing concentration of IL-2. FIG. 2. In addition, the fusion of IL-2 with the high affinity EBV IL-10 (internally termed DV07, Seq ID No: 5), which is known as DK2¹⁰EGFR, prevents the induction of IL-2 mediated secondary cytokines both from PBMC and from anti-CD3 stimulated PBMC. FIG. 2. Further investigation of whether coupling IL-10 to IL-2 indicates that the application of DK2¹⁰(EGFR) to mice prevents the induction of peripheral cytokines induced by IL-2 alone. FIG. 3. Furthermore, the treatment of non-human primates with DK2¹⁰ (EGFR) similarly does not lead to significant inductions of peripheral plasma cytokines. FIG. 4.

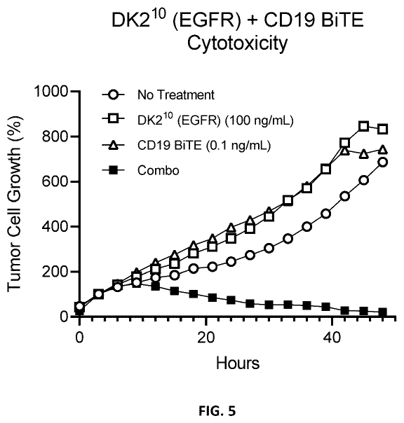

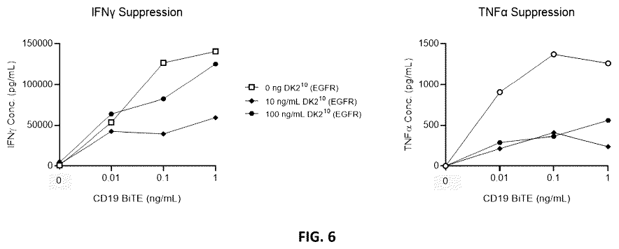

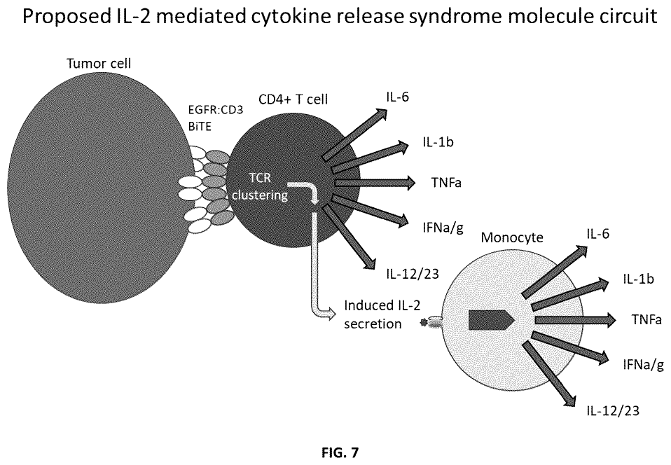

Moreover, pre-exposure of CD8+ T cells to a diakine (e.g., DK2¹⁰ (EGFR)) dramatically enhances the cells subsequent capacity to engage BiTE's and effect cytolysis of tumor cells. FIG. 5. Furthermore, these cells appear to secrete similar levels of IFNγ, but lower levels of TNFα compared to BiTE alone stimulated cells. FIG. 6. Thus, the inventor believes that the molecular circuit responsible for BiTE or CAR-T mediated CRS is a cascade of pro-inflammatory cytokine release, that is first triggered by CD4+ T cell engagement with BiTE or CAR-T, which subsequently triggers IL-2 secretion and inducement of monocyte/macrophage to undergo additional secretion of proinflammatory cytokines. FIG. 7 from monocytes/macrophages.

### Example 1: Diakine Enhances BiTE Mediated Tumor Cell Cytolysis

DK2¹⁰ (EGFR) has been previously shown to prime CD8+ T cell for subsequent tumor cytolysis in vitro and in vivo. See, U.S. Pat. No. 11,292,822. Here, DK2¹⁰ (EGFR), which is a representative example of a diakine, is shown to both enhance BiTE mediated tumor cell cytolysis as well as suppress BiTE mediated CRS.

Initially, DK2¹⁰ (EGFR) is shown to enhance BiTE mediated tumor cell cytotoxicity. In this in vitro model, the combinatorial anti-tumor effects of DK2¹⁰ (EGFR) with the CD19 BiTE is assessed in multiple rounds of exposure to target tumor cells.

CD8+ T cells are isolated from fresh donor Leukopaks via magnetic bead isolation per the manufacturer's suggested protocol (Miltenyi). The isolated CD8+ T cells are plated at 2.5×10⁶ cells/well and exposed for 2 days in various concentrations (0 or 100 ng/mL) of DK2¹⁰ (EGFR) in AIMV. Following the 2 days of exposure to the various concentrations of DK2¹⁰ (EGFR), the CD8+ T cells are harvested, counted, washed, and finally resuspended in the corresponding concentration of DK2¹⁰ (EGFR). Concurrently, Raji cells, which constitutively express Green Florescent Protein (GFP), are counted, washed and resuspended in varying concentrations (0 or 0.1 ng/mL) of CD19 BiTE. The CD8+ cells (effector) and Raji-GFP cells (target) are then combined at a 10:1 effector to target ratio. The mixture of effector and target cells, which are exposed to CD19 BiTE alone, DK2¹⁰ (EGFR) alone, or the combination of CD19 BiTE and DK2¹⁰ (EGFR), were monitored over 5 days using an IncuCyte® S3 Live-Cell Analysis System (Essen Bioscience/Sartorius). In conjunction, additional plates were seeded with the same conditions as stated above to be used for subsequent and serial rounds of the cytotoxicity assay. Every 3 days media is aspirated from wells and fresh media, with appropriate concentrations of either DK2¹⁰ (EGFR), CD19 BiTE, or the combination of the two, are added to the wells. Following the 5-day exposure, cells are harvested, counted, washed, and re-exposed to the similar conditions as stated previously. The percentage of (GFP) disappearance is measured as an indicator of cytotoxicity.

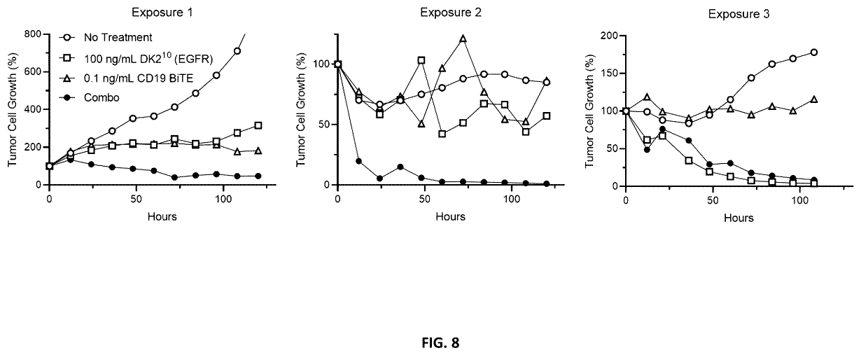

CD3×CD19 BiTE when combined with DK2¹⁰ (EGFR) enhances tumor cell cytotoxicity. See, FIGS. 5 and 8. CD3XCD19 BiTE and DK2¹⁰ (EGFR) are tested both alone and in combination using normal, healthy human donor derived CD8+ T cells. FIG. 8. Effector cells went through multiple rounds (5 rounds of serial cytolysis) of exposure to target tumor cells (Raji-GFP), and cytotoxicity was measured via the disappearance of GFP. In vitro treatment with the BiTE in combination with DK2¹⁰ (EGFR) suggests that activation of T cells with DK2¹⁰ (EGFR) enhances responses to BITE's when assessing CD8+ T cell anti-Raji^{GFP+} responses.

### Example 2: Diakine Reduces BiTE Mediated CRS

One of the current clinical challenges with BiTE's is the significant induction of CRS (Zhou, 2021). Since diakine (e.g., DK2¹⁰ (EGFR)) treatment of peripheral blood derived mononuclear cells (PBMC), tumor-bearing mice and non-human primates (see, FIGS. 1, 3, and 4) appears to prevent IL-2 mediated CRS, in vitro cultures comprising PBMC, Raji^{GFP+} cells, 0.1 ng/mL CD19 BiTE with or without 100 ng/mL DK2¹⁰ (EGFR) are used to determine whether DK2¹⁰ (EGFR) is capable of suppressing BiTE mediated CRS.

PBMCs are isolated from Leukopaks collected from healthy donors using the Ficoll density gradient method. Equal volumes of HBSS and donor samples were transferred to conical tubes, individually. Ficoll was slowly added to create a bottom layer then samples were centrifuged at 400×g for 30 minutes at 25° C. PBMCs were harvested from the top layer then washed twice using Aim V media (300×g, 8 minutes). The isolated PBMCs were plated at 2×10⁶ cells/well in various concentrations (0 or 100 ng/mL) of DK2¹⁰ EGFR in AIMV then incubated for two days (37° C., 5% CO₂). Raji tumor cells were counted, washed and resuspended in varying concentrations of CD19 BiTE (0, or 0.1 ng/mL final), with or without DK2¹⁰ (EGFR) (100 ng/mL) using AIMV and allowed to prime for two-days. The PBMCs, assuming 10% CD8+ T cells (effector), and Raji cells (target) were combined at a 10:1 effector to target ratio. After 24-hour incubation, supernatants are harvested and cytokine secretion was measured via multiplexed capture assay (MSD) and ELISA.

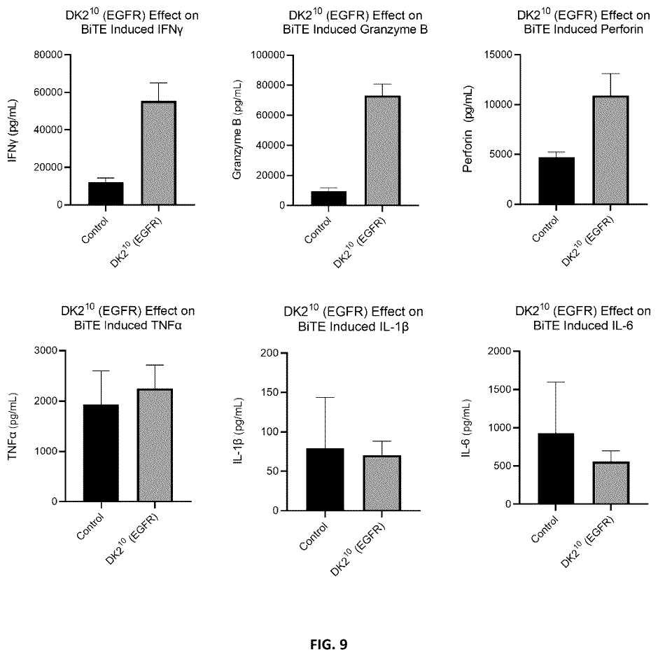

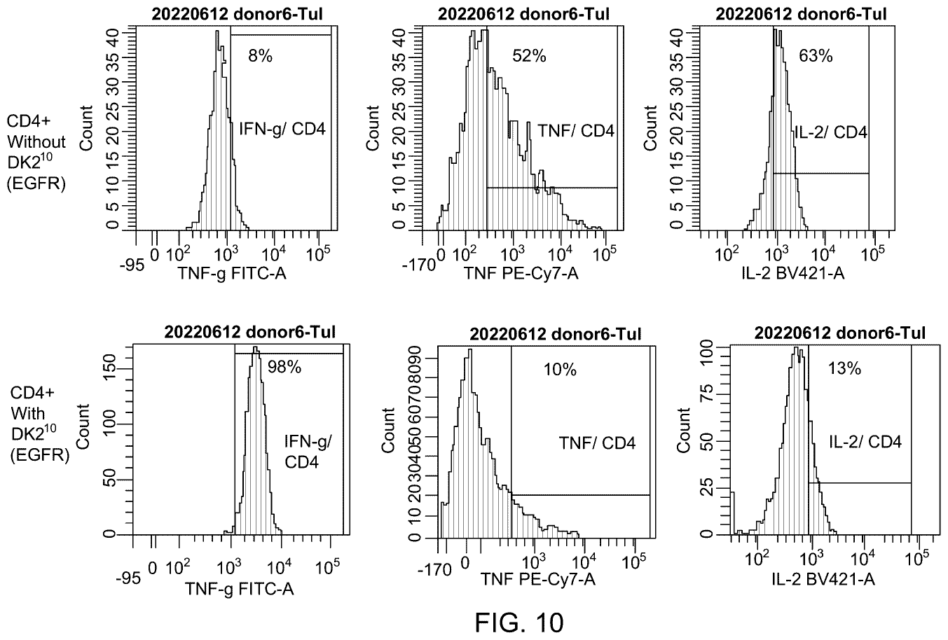

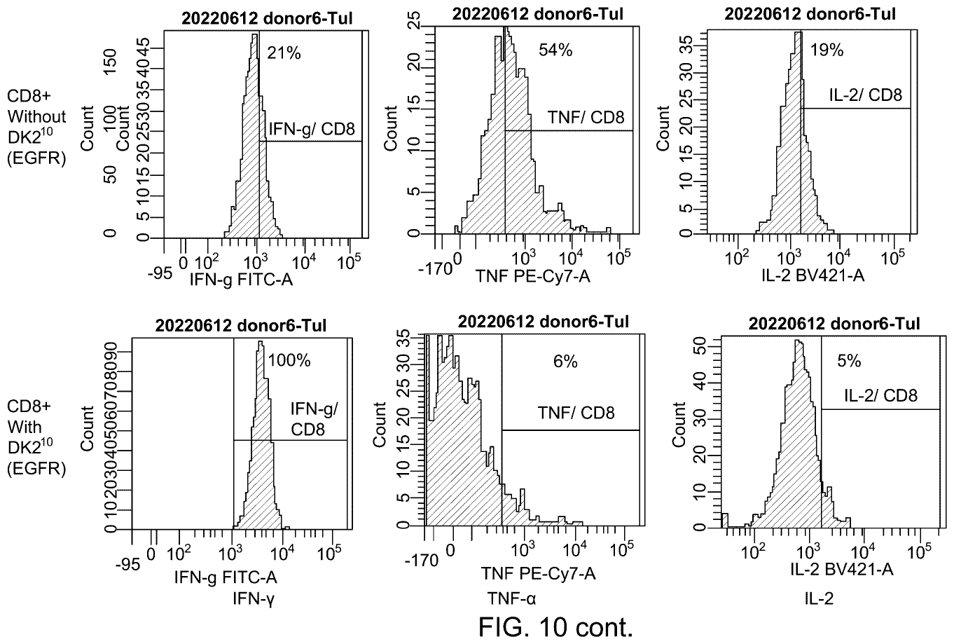

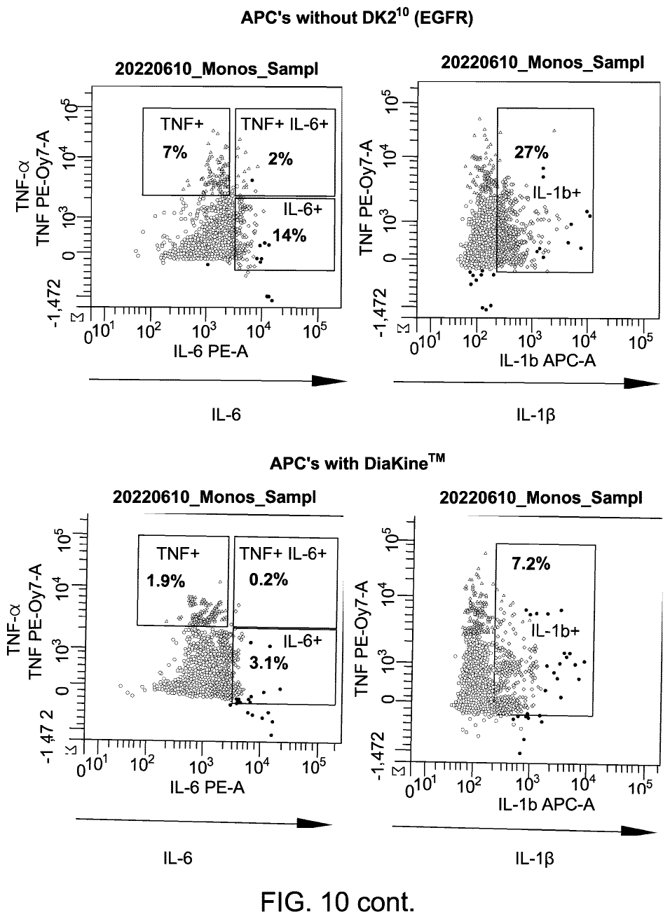

Illustrated in FIG. 9, the data suggests that the presence of DK2¹⁰ (EGFR) under these conditions significantly promotes tumor cell lysis (induction of IFN-gamma, Granzyme B, and Perforin) while limiting the induction of BiTE mediated CRS at a non-functional concentration of BiTE (0.1 ng/mL). Represented data is taken at 24 hrs., from exposure 1. Longitudinal data indicates a reduction of CRS over time in these conditions. FIG. 10.

To better understand the cell types present in the PBMC+Raji^{GFP+} cultures that are affected by DK2¹⁰ (EGFR), we assessed 48-hour cultures by intracellular fluorescence activated cell sorting (FACS). This analysis, suggests that the presence of DK2¹⁰ (EGFR) polarizes both CD4+ and CD8+ T cells to predominantly express IFN-gamma, while reducing TNFalpha and IL-2 production. MHC II positive cells, denoted as antigen-presenting cells (APC's), also exhibit reduced TNFalpha, IL-6 and IL-1-beta production, suggesting DK2¹⁰ (EGFR) mediates pleiotropic cell type control of CRS associated with BiTE mediated T cell activation. FIG. 10.

### REFERENCES

BIBLIOGRAPHY Bosco, M. C. (2000). 11-2 Signaling in Human Monocytes Involves the Phosphorylation and Activation of p59hck1. Journal of Immunology.
Brandl, C. (2007). The effect of dexamethasone on polyclonal T cell activation and redirected target cell lysis as induced by a CD19 CD3 bispecific single-chain antibody construct. Cancer Immunology Immunotherapy.
Chen, Y. (2021). Therapeutic Potential of TNFa and IL-1b Blockade for CRS/ICANS in CART Therapy via Ameliorating Endothelial Activation. Frontiers in Immunology.
Hosseini, I. (2020). Mitigating the risk of cytokine release syndrome in Phase I trial of CD20CD3 bispecific antibody mosunetuzumab in NHL impact of translational system modeling. Systems Biology and Applications.
Fu et al (1029). Therapeutic Bispecific T-Cell Engager Antibody Targeting the Transferrin Receptor. Frontiers in Immunology.
Liu, D. (2018). Cytokine release syndrome; grading, modeling, and new therapy. Journal of Hematology and Oncology.
Maude, S. L. (2014). Managing Cytokine Release Syndrome Associated with Novel T Cell-Engaging Therapies. Cancer Journal.
Musso, T. (1992). IL-2 Induces IL-6 Production in Human Monocytes. Journal of Immunology.
Norelli, M. (2018). Monocyte-derived IL-1 and IL-6 are differentially required for cytokine release syndrome and neurotoxicity due to CAR T cells. Nature Medicine.
Shimabukuro-Vornhagen, A. (2018). Cytokine release syndrome. Journal of Immuno Therapy of Cancer.
Strieter, R. M. (1989). Interleukin-2-induced Tumor Necrosis Factor-alpha Gene Expression in Human Alveolar Macrophages and Blood Monocytes. American Review Respiratory Disease.

1. A method of reducing the severity of bispecific T cell engager (BiTE) or chimeric antigen receptor T cell (CAR-T) therapy induced cytokine release syndrome (CRS) during malignant disease treatment and enhancing tumor cytolysis comprising administering to a patient in need thereof an amount of a composition comprising a dual cytokine fusion protein of Formula II
NH₂-(Y)-(X¹)-(Zn)-(X²)-(Y)-COOH (Formula II)
wherein
“Y” is a monomer of IL10 or an variant thereof;
“X” is a VL or VH region from a first monoclonal antibody;
“X²” is a VH or VL region from the first monoclonal antibody;
wherein when X¹ is a VL, X² is a VH or when X¹ is a VH, X² is a VL,
wherein the first monoclonal antibody is an anti-ebola antibody;
wherein the VL and VH from the anti-ebola antibody include 3 light chain CDRs and 3 heavy chain CDRs that are engrafted with 3 light chain CDRs and 3 heavy chain CDRs from a second monoclonal antibody;
“Z” is a cytokine other than IL-10;
“n” is an integer of 1; and
wherein:
i. the IL-10 is SEQ ID No: 1, the second antibody is an anti-epidermal growth factor receptor (EGFR) monoclonal antibody, and Z is IL-2;
ii. the IL-10 is SEQ ID No: 1, the second antibody is an anti-EGFR monoclonal antibody, and Z is IL-15;
iii. the IL-10 is SEQ ID No: 1, the second antibody is an anti-EGFR monoclonal antibody, and Z is IL-7;
iv. the IL-10 is SEQ ID No: 1, the second antibody is an anti-EGFR monoclonal antibody, and Z is IL-28;
v. the IL-10 is SEQ ID No: 1, the second antibody is an anti-EGFR monoclonal antibody, and Z is IL-29;
vi. the IL-10 is SEQ ID No: 1, the second antibody is an anti-EGFR monoclonal antibody, and Z is IFN-α;
vii. the IL-10 is SEQ ID No: 1, the second antibody is an anti-EGFR monoclonal antibody, and Z is IL-21;
viii. the IL-10 is SEQ ID No: 1, the second antibody is an anti-HER2 monoclonal antibody, and Z is IL-2;
ix. the IL-10 is SEQ ID No: 1, the second antibody is an anti-HER2 monoclonal antibody, and Z is IL-15;
x. the IL-10 is SEQ ID No: 1, the second antibody is an anti-HER2 monoclonal antibody, and Z is IL-7;
xi. the IL-10 is SEQ ID No: 1, the second antibody is an anti-HER2 monoclonal antibody, and Z is IL-28;
xii. the IL-10 is SEQ ID No: 1, the second antibody is an anti-HER2 monoclonal antibody, and Z is IL-29;
xiii. the IL-10 is SEQ ID No: 1, the second antibody is an anti-HER2 monoclonal antibody, and Z is IFN-α;
xiv. the IL-10 is SEQ ID No: 1, the second antibody is an anti-HER2 monoclonal antibody, and Z is IL-21;
xv. the IL-10 is SEQ ID No: 1, the second antibody is an anti-VEGFR2 monoclonal antibody, and Z is IL-2;
xvi. the IL-10 is SEQ ID No: 1, the second antibody is an anti-VEGFR2 monoclonal antibody, and Z is IL-15;
xvii. the IL-10 is SEQ ID No: 1, the second antibody is an anti-VEGFR2 monoclonal antibody, and Z is IL-7;
xviii. the IL-10 is SEQ ID No: 1, the second antibody is an anti-VEGFR2 monoclonal antibody, and Z is IL-28;
xix. the IL-10 is SEQ ID No: 1, the second antibody is an anti-VEGFR2 monoclonal antibody, and Z is IL-29;
xx. the IL-10 is SEQ ID No: 1, the second antibody is an anti-VEGFR2 monoclonal antibody, and Z is IFN-α;
xxi. the IL-10 is SEQ ID No: 1, the second antibody is an anti-VEGFR2 monoclonal antibody, and Z is IL-21;
xxii. the IL-10 is SEQ ID No: 1, the second antibody is an anti-PDGFR monoclonal antibody, and Z is IL-2;
xxiii. the IL-10 is SEQ ID No: 1, the second antibody is an anti-PDGFR monoclonal antibody, and Z is IL-15;
xxiv. the IL-10 is SEQ ID No: 1, the second antibody is an anti-PDGFR monoclonal antibody, and Z is IL-7;
xxv. the IL-10 is SEQ ID No: 1, the second antibody is an anti-PDGFR monoclonal antibody, and Z is IL-28;
xxvi. the IL-10 is SEQ ID No: 1, the second antibody is an anti-PDGFR monoclonal antibody, and Z is IL-29;
xxvii. the IL-10 is SEQ ID No: 1, the second antibody is an anti-PDGFR monoclonal antibody, and Z is IFN-α;
xxviii. the IL-10 is SEQ ID No: 1, the second antibody is an anti-PDGFR monoclonal antibody, and Z is IL-21;
xxix. the IL-10 is SEQ ID No: 1, the second antibody is an anti-GPC3 monoclonal antibody, and Z is IL-2;
xxx. the IL-10 is SEQ ID No: 1, the second antibody is an anti-GPC3 monoclonal antibody, and Z is IL-15;
xxxi. the IL-10 is SEQ ID No: 1, the second antibody is an anti-GPC3 monoclonal antibody, and Z is IL-7;
xxxii. the IL-10 is SEQ ID No: 1, the second antibody is an anti-GPC3 monoclonal antibody, and Z is IL-28;
xxxiii. the IL-10 is SEQ ID No: 1, the second antibody is an anti-GPC3 monoclonal antibody, and Z is IL-29;
xxxiv. the IL-10 is SEQ ID No: 1, the second antibody is an anti-GPC3 monoclonal antibody, and Z is IFN-α;
xxxv. the IL-10 is SEQ ID No: 1, the second antibody is an anti-GPC3 monoclonal antibody, and Z is IL-21;
xxxvi. the IL-10 is SEQ ID No: 1, the second antibody is an anti-PD-L1 monoclonal antibody, and Z is IL-2;
xxxvii. the IL-10 is SEQ ID No: 1, the second antibody is an anti-PD-L1 monoclonal antibody, and Z is IL-15;
xxxviii. the IL-10 is SEQ ID No: 1, the second antibody is an anti-PD-L1 monoclonal antibody, and Z is IL-7;
xxxix. the IL-10 is SEQ ID No: 1, the second antibody is an anti-PD-L1 monoclonal antibody, and Z is IL-28;
xl. the IL-10 is SEQ ID No: 1, the second antibody is an anti-PD-L1 monoclonal antibody, and Z is IL-29;
xli. the IL-10 is SEQ ID No: 1, the second antibody is an anti-PD-L1 monoclonal antibody, and Z is IFN-α;
xlii. the IL-10 is SEQ ID No: 1, the second antibody is an anti-PD-L1 monoclonal antibody, and Z is IL-21;
xliii. the IL-10 is SEQ ID No: 1, the second antibody is an anti-CD19 monoclonal antibody, and Z is IL-2;
xliv. the IL-10 is SEQ ID No: 1, the second antibody is an anti-CD19 monoclonal antibody, and Z is IL-15;
xlv. the IL-10 is SEQ ID No: 1, the second antibody is an anti-CD19 monoclonal antibody, and Z is IL-7;
xlvi. the IL-10 is SEQ ID No: 1, the second antibody is an anti-CD19 monoclonal antibody, and Z is IL-28;
xlvii. the IL-10 is SEQ ID No: 1, the second antibody is an anti-CD19 monoclonal antibody, and Z is IL-29;
xlviii. the IL-10 is SEQ ID No: 1, the second antibody is an anti-CD19 monoclonal antibody, and Z is IFN-α;
xlix. the IL-10 is SEQ ID No: 1, the second antibody is an anti-CD19 monoclonal antibody, and Z is IL-21;
l. the IL-10 is SEQ ID No: 1, the second antibody is an anti-CD20 monoclonal antibody, and Z is IL-2;
li. the IL-10 is SEQ ID No: 1, the second antibody is an anti-CD20 monoclonal antibody, and Z is IL-15;
lii. the IL-10 is SEQ ID No: 1, the second antibody is an anti-CD20 monoclonal antibody, and Z is IL-7;
liii. the IL-10 is SEQ ID No: 1, the second antibody is an anti-CD20 monoclonal antibody, and Z is IL-28;
liv. the IL-10 is SEQ ID No: 1, the second antibody is an anti-CD20 monoclonal antibody, and Z is IL-29;
lv. the IL-10 is SEQ ID No: 1, the second antibody is an anti-CD20 monoclonal antibody, and Z is IFN-α;
lvi. the IL-10 is SEQ ID No: 1, the second antibody is an anti-CD20 monoclonal antibody, and Z is IL-21;
lvii. the IL-10 is SEQ ID No: 1, the second antibody is an anti-CD22 monoclonal antibody, and Z is IL-2;
lviii. the IL-10 is SEQ ID No: 1, the second antibody is an anti-CD22 monoclonal antibody, and Z is IL-15;
lix. the IL-10 is SEQ ID No: 1, the second antibody is an anti-CD22 monoclonal antibody, and Z is IL-7;
lx. the IL-10 is SEQ ID No: 1, the second antibody is an anti-CD22 monoclonal antibody, and Z is IL-28;
lxi. the IL-10 is SEQ ID No: 1, the second antibody is an anti-CD22 monoclonal antibody, and Z is IL-29;
lxii. the IL-10 is SEQ ID No: 1, the second antibody is an anti-CD22 monoclonal antibody, and Z is IFN-α;
lxiii. the IL-10 is SEQ ID No: 1, the second antibody is an anti-CD22 monoclonal antibody, and Z is IL-21;
lxiv. the IL-10 is SEQ ID No: 1, the second antibody is an anti-PSMA monoclonal antibody, and Z is IL-2;
lxv. the IL-10 is SEQ ID No: 1, the second antibody is an anti-PSMA monoclonal antibody, and Z is IL-15;
lxvi. the IL-10 is SEQ ID No: 1, the second antibody is an anti-PSMA monoclonal antibody, and Z is IL-7;
lxvii. the IL-10 is SEQ ID No: 1, the second antibody is an anti-PSMA monoclonal antibody, and Z is IL-28;
lxviii. the IL-10 is SEQ ID No: 1, the second antibody is an anti-PSMA monoclonal antibody, and Z is IL-29;
lxix. the IL-10 is SEQ ID No: 1, the second antibody is an anti-PSMA monoclonal antibody, and Z is IFN-α;
lxx. the IL-10 is SEQ ID No: 1, the second antibody is an anti-PSMA monoclonal antibody, and Z is IL-21;
lxxi. the IL-10 is SEQ ID No: 1, the second antibody is an anti-CEA monoclonal antibody, and Z is IL-2;
lxxii. the IL-10 is SEQ ID No: 1, the second antibody is an anti-CEA monoclonal antibody, and Z is IL-15;
lxxiii. the IL-10 is SEQ ID No: 1, the second antibody is an anti-CEA monoclonal antibody, and Z is IL-7;
lxxiv. the IL-10 is SEQ ID No: 1, the second antibody is an anti-CEA monoclonal antibody, and Z is IL-28;
lxxv. the IL-10 is SEQ ID No: 1, the second antibody is an anti-CEA monoclonal antibody, and Z is IL-29;
lxxvi. the IL-10 is SEQ ID No: 1, the second antibody is an anti-CEA monoclonal antibody, and Z is IFN-α;
lxxvii. the IL-10 is SEQ ID No: 1, the second antibody is an anti-CEA monoclonal antibody, and Z is IL-21;
lxxviii. the IL-10 is SEQ ID No: 1, the second antibody is an anti-BCMA monoclonal antibody, and Z is IL-2;
lxxix. the IL-10 is SEQ ID No: 1, the second antibody is an anti-BCMA monoclonal antibody, and Z is IL-15;
lxxx. the IL-10 is SEQ ID No: 1, the second antibody is an anti-BCMA monoclonal antibody, and Z is IL-7;
lxxxi. the IL-10 is SEQ ID No: 1, the second antibody is an anti-BCMA monoclonal antibody, and Z is IL-28;
lxxxii. the IL-10 is SEQ ID No: 1, the second antibody is an anti-BCMA monoclonal antibody, and Z is IL-29;
lxxxiii. the IL-10 is SEQ ID No: 1, the second antibody is an anti-BCMA monoclonal antibody, and Z is IFN-α;
lxxxiv. the IL-10 is SEQ ID No: 1, the second antibody is an anti-BCMA monoclonal antibody, and Z is IL-21;
lxxxv. the IL-10 is SEQ ID No: 1, the second antibody is an anti-gpA33 monoclonal antibody, and Z is IL-2;
lxxxvi. the IL-10 is SEQ ID No: 1, the second antibody is an anti-gpA33 monoclonal antibody, and Z is IL-15;
lxxxvii. the IL-10 is SEQ ID No: 1, the second antibody is an anti-gpA33 monoclonal antibody, and Z is IL-7;
lxxxviii. the IL-10 is SEQ ID No: 1, the second antibody is an anti-gpA33 monoclonal antibody, and Z is IL-28;
lxxxix. the IL-10 is SEQ ID No: 1, the second antibody is an anti-gpA33 monoclonal antibody, and Z is IL-29;
xc. the IL-10 is SEQ ID No: 1, the second antibody is an anti-gpA33 monoclonal antibody, and Z is IFN-α;
xci. the IL-10 is SEQ ID No: 1, the second antibody is an anti-gpA33 monoclonal antibody, and Z is IL-21;
xcii. the IL-10 is SEQ ID No: 1, the second antibody is an anti-CD33 monoclonal antibody, and Z is IL-2;
xciii. the IL-10 is SEQ ID No: 1, the second antibody is an anti-CD33 monoclonal antibody, and Z is IL-15;
xciv. the IL-10 is SEQ ID No: 1, the second antibody is an anti-CD33monoclonal antibody, and Z is IL-7;
xcv. the IL-10 is SEQ ID No: 1, the second antibody is an anti-CD33 monoclonal antibody, and Z is IL-28;
xcvi. the IL-10 is SEQ ID No: 1, the second antibody is an anti-CD33 monoclonal antibody, and Z is IL-29;
xcvii. the IL-10 is SEQ ID No: 1, the second antibody is an anti-CD33 monoclonal antibody, and Z is IFN-α;
xcviii. the IL-10 is SEQ ID No: 1, the second antibody is an anti-CD33 monoclonal antibody, and Z is IL-21;
xcix. the IL-10 is SEQ ID No: 1, the second antibody is an anti-DLL3 monoclonal antibody, and Z is IL-2;
c. the IL-10 is SEQ ID No: 1, the second antibody is an anti-DLL3 monoclonal antibody, and Z is IL-15;
ci. the IL-10 is SEQ ID No: 1, the second antibody is an anti-DLL3 monoclonal antibody, and Z is IL-7;
cii. the IL-10 is SEQ ID No: 1, the second antibody is an anti-DLL3 monoclonal antibody, and Z is IL-28;
ciii. the IL-10 is SEQ ID No: 1, the second antibody is an anti-DLL3 monoclonal antibody, and Z is IL-29;
civ. the IL-10 is SEQ ID No: 1, the second antibody is an anti-DLL3 monoclonal antibody, and Z is IFN-α;
cv. the IL-10 is SEQ ID No: 1, the second antibody is an anti-DLL3 monoclonal antibody, and Z is IL-21;
cvi. the IL-10 is SEQ ID No: 1, the second antibody is an anti-MUC17 monoclonal antibody, and Z is IL-2;
cvii. the IL-10 is SEQ ID No: 1, the second antibody is an anti-MUC17 monoclonal antibody, and Z is IL-15;
cviii. the IL-10 is SEQ ID No: 1, the second antibody is an anti-MUC17 monoclonal antibody, and Z is IL-7;
cix. the IL-10 is SEQ ID No: 1, the second antibody is an anti-MUC17 monoclonal antibody, and Z is IL-28;
cx. the IL-10 is SEQ ID No: 1, the second antibody is an anti-MUC17 monoclonal antibody, and Z is IL-29;
cxi. the IL-10 is SEQ ID No: 1, the second antibody is an anti-MUC17 monoclonal antibody, and Z is IFN-α;
cxii. the IL-10 is SEQ ID No: 1, the second antibody is an anti-MUC17 monoclonal antibody, and Z is IL-21;
cxiii. the IL-10 is SEQ ID No: 1, the second antibody is an anti-CLDN18 monoclonal antibody, and Z is IL-2;
cxiv. the IL-10 is SEQ ID No: 1, the second antibody is an anti-CLDN18 monoclonal antibody, and Z is IL-15;
cxv. the IL-10 is SEQ ID No: 1, the second antibody is an anti-CLDN18 monoclonal antibody, and Z is IL-7;
cxvi. the IL-10 is SEQ ID No: 1, the second antibody is an anti-CLDN18 monoclonal antibody, and Z is IL-28;
cxvii. the IL-10 is SEQ ID No: 1, the second antibody is an anti-CLDN18 monoclonal antibody, and Z is IL-29;
cxviii. the IL-10 is SEQ ID No: 1, the second antibody is an anti-CLDN18 monoclonal antibody, and Z is IFN-α;
cxix. the IL-10 is SEQ ID No: 1, the second antibody is an anti-CLDN18 monoclonal antibody, and Z is IL-21;
cxx. the IL-10 is SEQ ID No: 1, the second antibody is an anti-GD2 monoclonal antibody, and Z is IL-2;
cxxi. the IL-10 is SEQ ID No: 1, the second antibody is an anti-GD2 monoclonal antibody, and Z is IL-15;
cxxii. the IL-10 is SEQ ID No: 1, the second antibody is an anti-GD2 monoclonal antibody, and Z is IL-7;
cxxiii. the IL-10 is SEQ ID No: 1, the second antibody is an anti-GD2 monoclonal antibody, and Z is IL-28;
cxxiv. the IL-10 is SEQ ID No: 1, the second antibody is an anti-GD2 monoclonal antibody, and Z is IL-29;
cxxv. the IL-10 is SEQ ID No: 1, the second antibody is an anti-GD2 monoclonal antibody, and Z is IFN-α;
cxxvi. the IL-10 is SEQ ID No: 1, the second antibody is an anti-GD2 monoclonal antibody, and Z is IL-21;
cxxvii. the IL-10 is SEQ ID No: 1, the second antibody is an anti-5T4 monoclonal antibody, and Z is IL-2;
cxxviii. the IL-10 is SEQ ID No: 1, the second antibody is an anti-5T4 monoclonal antibody, and Z is IL-15;
cxxix. the IL-10 is SEQ ID No: 1, the second antibody is an anti-5T4 monoclonal antibody, and Z is IL-7;
cxxx. the IL-10 is SEQ ID No: 1, the second antibody is an anti-5T4 monoclonal antibody, and Z is IL-28;
cxxxi. the IL-10 is SEQ ID No: 1, the second antibody is an anti-5T4 monoclonal antibody, and Z is IL-29;
cxxxii. the IL-10 is SEQ ID No: 1, the second antibody is an anti-5T4 monoclonal antibody, and Z is IFN-α;
cxxxiii. the IL-10 is SEQ ID No: 1, the second antibody is an anti-5T4 monoclonal antibody, and Z is IL-21;
cxxxiv. the IL-10 is SEQ ID No: 1, the second antibody is an anti-HER3 monoclonal antibody, and Z is IL-2;
cxxxv. the IL-10 is SEQ ID No: 1, the second antibody is an anti-HER3 monoclonal antibody, and Z is IL-15;
cxxxvi. the IL-10 is SEQ ID No: 1, the second antibody is an anti-HER3 monoclonal antibody, and Z is IL-7;
cxxxvii. the IL-10 is SEQ ID No: 1, the second antibody is an anti-HER3 monoclonal antibody, and Z is IL-28;
cxxxviii. the IL-10 is SEQ ID No: 1, the second antibody is an anti-HER3 monoclonal antibody, and Z is IL-29;
cxxxix. the IL-10 is SEQ ID No: 1, the second antibody is an anti-HER3 monoclonal antibody, and Z is IFN-α;
cxl. the IL-10 is SEQ ID No: 1, the second antibody is an anti-HER3 monoclonal antibody, and Z is IL-21;
cxli. the IL-10 is SEQ ID No: 5, the second antibody is an anti-EGFR monoclonal antibody, and Z is IL-2;
cxlii. the IL-10 is SEQ ID No: 5, the second antibody is an anti-EGFR monoclonal antibody, and Z is IL-15;
cxliii. the IL-10 is SEQ ID No: 5, the second antibody is an anti-EGFR monoclonal antibody, and Z is IL-7;
cxliv. the IL-10 is SEQ ID No: 5, the second antibody is an anti-EGFR monoclonal antibody, and Z is IL-28;
cxlv. the IL-10 is SEQ ID No: 5, the second antibody is an anti-EGFR monoclonal antibody, and Z is IL-29;
cxlvi. the IL-10 is SEQ ID No: 5, the second antibody is an anti-EGFR monoclonal antibody, and Z is IFN-α;
cxlvii. the IL-10 is SEQ ID No: 5, the second antibody is an anti-EGFR monoclonal antibody, and Z is IL-21;
cxlviii. the IL-10 is SEQ ID No: 5, the second antibody is an anti-HER2 monoclonal antibody, and Z is IL-2;
cxlix. the IL-10 is SEQ ID No: 5, the second antibody is an anti-HER2 monoclonal antibody, and Z is IL-15;
cl. the IL-10 is SEQ ID No: 5, the second antibody is an anti-HER2 monoclonal antibody, and Z is IL-7;
cli. the IL-10 is SEQ ID No: 5, the second antibody is an anti-HER2 monoclonal antibody, and Z is IL-28;
clii. the IL-10 is SEQ ID No: 5, the second antibody is an anti-HER2 monoclonal antibody, and Z is IL-29;
cliii. the IL-10 is SEQ ID No: 5, the second antibody is an anti-HER2 monoclonal antibody, and Z is IFN-α;
cliv. the IL-10 is SEQ ID No: 5, the second antibody is an anti-HER2 monoclonal antibody, and Z is IL-21;
clv. the IL-10 is SEQ ID No: 5, the second antibody is an anti-VEGFR2 monoclonal antibody, and Z is IL-2;
clvi. the IL-10 is SEQ ID No: 5, the second antibody is an anti-VEGFR2 monoclonal antibody, and Z is IL-15;
clvii. the IL-10 is SEQ ID No: 5, the second antibody is an anti-VEGFR2 monoclonal antibody, and Z is IL-7;
clviii. the IL-10 is SEQ ID No: 5, the second antibody is an anti-VEGFR2 monoclonal antibody, and Z is IL-28;
clix. the IL-10 is SEQ ID No: 5, the second antibody is an anti-VEGFR2 monoclonal antibody, and Z is IL-29;
clx. the IL-10 is SEQ ID No: 5, the second antibody is an anti-VEGFR2 monoclonal antibody, and Z is IFN-α;
clxi. the IL-10 is SEQ ID No: 5, the second antibody is an anti-VEGFR2 monoclonal antibody, and Z is IL-21;
clxii. the IL-10 is SEQ ID No: 5, the second antibody is an anti-PDGFR monoclonal antibody, and Z is IL-2;
clxiii. the IL-10 is SEQ ID No: 5, the second antibody is an anti-PDGFR monoclonal antibody, and Z is IL-15;
clxiv. the IL-10 is SEQ ID No: 5, the second antibody is an anti-PDGFR monoclonal antibody, and Z is IL-7;
clxv. the IL-10 is SEQ ID No: 5, the second antibody is an anti-PDGFR monoclonal antibody, and Z is IL-28;
clxvi. the IL-10 is SEQ ID No: 5, the second antibody is an anti-PDGFR monoclonal antibody, and Z is IL-29;
clxvii. the IL-10 is SEQ ID No: 5, the second antibody is an anti-PDGFR monoclonal antibody, and Z is IFN-α;
clxviii. the IL-10 is SEQ ID No: 5, the second antibody is an anti-PDGFR monoclonal antibody, and Z is IL-21;
clxix. the IL-10 is SEQ ID No: 5, the second antibody is an anti-GPC3 monoclonal antibody, and Z is IL-2;
clxx. the IL-10 is SEQ ID No: 5, the second antibody is an anti-GPC3 monoclonal antibody, and Z is IL-15;
clxxi. the IL-10 is SEQ ID No: 5, the second antibody is an anti-GPC3 monoclonal antibody, and Z is IL-7;
clxxii. the IL-10 is SEQ ID No: 5, the second antibody is an anti-GPC3 monoclonal antibody, and Z is IL-28;
clxxiii. the IL-10 is SEQ ID No: 5, the second antibody is an anti-GPC3 monoclonal antibody, and Z is IL-29;
clxxiv. the IL-10 is SEQ ID No: 5, the second antibody is an anti-GPC3 monoclonal antibody, and Z is IFN-α;
clxxv. the IL-10 is SEQ ID No: 5, the second antibody is an anti-GPC3 monoclonal antibody, and Z is IL-21;
clxxvi. the IL-10 is SEQ ID No: 5, the second antibody is an anti-PD-L1 monoclonal antibody, and Z is IL-2;
clxxvii. the IL-10 is SEQ ID No: 5, the second antibody is an anti-PD-L1 monoclonal antibody, and Z is IL-15;
clxxviii. the IL-10 is SEQ ID No: 5, the second antibody is an anti-PD-L1 monoclonal antibody, and Z is IL-7;
clxxix. the IL-10 is SEQ ID No: 5, the second antibody is an anti-PD-L1 monoclonal antibody, and Z is IL-28;
clxxx. the IL-10 is SEQ ID No: 5, the second antibody is an anti-PD-L1 monoclonal antibody, and Z is IL-29;
clxxxi. the IL-10 is SEQ ID No: 5, the second antibody is an anti-PD-L1 monoclonal antibody, and Z is IFN-α;
clxxxii. the IL-10 is SEQ ID No: 5, the second antibody is an anti-PD-L1 monoclonal antibody, and Z is IL-21;
clxxxiii. the IL-10 is SEQ ID No: 5, the second antibody is an anti-CD19 monoclonal antibody, and Z is IL-2;
clxxxiv. the IL-10 is SEQ ID No: 5, the second antibody is an anti-CD19 monoclonal antibody, and Z is IL-15;
clxxxv. the IL-10 is SEQ ID No: 5, the second antibody is an anti-CD19 monoclonal antibody, and Z is IL-7;
clxxxvi. the IL-10 is SEQ ID No: 5, the second antibody is an anti-CD19 monoclonal antibody, and Z is IL-28;
clxxxvii. the IL-10 is SEQ ID No: 5, the second antibody is an anti-CD19 monoclonal antibody, and Z is IL-29;
clxxxviii. the IL-10 is SEQ ID No: 5, the second antibody is an anti-CD19 monoclonal antibody, and Z is IFN-α;
clxxxix. the IL-10 is SEQ ID No: 5, the second antibody is an anti-CD19 monoclonal antibody, and Z is IL-21;
cxc. the IL-10 is SEQ ID No: 5, the second antibody is an anti-CD20 monoclonal antibody, and Z is IL-2;
cxci. the IL-10 is SEQ ID No: 5, the second antibody is an anti-CD20 monoclonal antibody, and Z is IL-15;
cxcii. the IL-10 is SEQ ID No: 5, the second antibody is an anti-CD20 monoclonal antibody, and Z is IL-7;
cxciii. the IL-10 is SEQ ID No: 5, the second antibody is an anti-CD20 monoclonal antibody, and Z is IL-28;
cxciv. the IL-10 is SEQ ID No: 5, the second antibody is an anti-CD20 monoclonal antibody, and Z is IL-29;
cxcv. the IL-10 is SEQ ID No: 5, the second antibody is an anti-CD20 monoclonal antibody, and Z is IFN-α;
cxcvi. the IL-10 is SEQ ID No: 5, the second antibody is an anti-CD20 monoclonal antibody, and Z is IL-21;
cxcvii. the IL-10 is SEQ ID No: 5, the second antibody is an anti-CD22 monoclonal antibody, and Z is IL-2;
cxcviii. the IL-10 is SEQ ID No: 5, the second antibody is an anti-CD22 monoclonal antibody, and Z is IL-15;
cxcix. the IL-10 is SEQ ID No: 5, the second antibody is an anti-CD22 monoclonal antibody, and Z is IL-7;
cc. the IL-10 is SEQ ID No: 5, the second antibody is an anti-CD22 monoclonal antibody, and Z is IL-28;
cci. the IL-10 is SEQ ID No: 5, the second antibody is an anti-CD22 monoclonal antibody, and Z is IL-29;
ccii. the IL-10 is SEQ ID No: 5, the second antibody is an anti-CD22 monoclonal antibody, and Z is IFN-α;
cciii. the IL-10 is SEQ ID No: 5, the second antibody is an anti-CD22 monoclonal antibody, and Z is IL-21;
cciv. the IL-10 is SEQ ID No: 5, the second antibody is an anti-PSMA monoclonal antibody, and Z is IL-2;
ccv. the IL-10 is SEQ ID No: 5, the second antibody is an anti-PSMA monoclonal antibody, and Z is IL-15;
ccvi. the IL-10 is SEQ ID No: 5, the second antibody is an anti-PSMA monoclonal antibody, and Z is IL-7;
ccvii. the IL-10 is SEQ ID No: 5, the second antibody is an anti-PSMA monoclonal antibody, and Z is IL-28;
ccviii. the IL-10 is SEQ ID No: 5, the second antibody is an anti-PSMA monoclonal antibody, and Z is IL-29;
ccix. the IL-10 is SEQ ID No: 5, the second antibody is an anti-PSMA monoclonal antibody, and Z is IFN-α;
ccx. the IL-10 is SEQ ID No: 5, the second antibody is an anti-PSMA monoclonal antibody, and Z is IL-21;
ccxi. the IL-10 is SEQ ID No: 5, the second antibody is an anti-CEA monoclonal antibody, and Z is IL-2;
ccxii. the IL-10 is SEQ ID No: 5, the second antibody is an anti-CEA monoclonal antibody, and Z is IL-15;
ccxiii. the IL-10 is SEQ ID No: 5, the second antibody is an anti-CEA monoclonal antibody, and Z is IL-7;
ccxiv. the IL-10 is SEQ ID No: 5, the second antibody is an anti-CEA monoclonal antibody, and Z is IL-28;
ccxv. the IL-10 is SEQ ID No: 5, the second antibody is an anti-CEA monoclonal antibody, and Z is IL-29;
ccxvi. the IL-10 is SEQ ID No: 5, the second antibody is an anti-CEA monoclonal antibody, and Z is IFN-α;
ccxvii. the IL-10 is SEQ ID No: 5, the second antibody is an anti-CEA monoclonal antibody, and Z is IL-21;
ccxviii. the IL-10 is SEQ ID No: 5, the second antibody is an anti-BCMA monoclonal antibody, and Z is IL-2;
ccxix. the IL-10 is SEQ ID No: 5, the second antibody is an anti-BCMA monoclonal antibody, and Z is IL-15;
ccxx. the IL-10 is SEQ ID No: 5, the second antibody is an anti-BCMA monoclonal antibody, and Z is IL-7;
ccxxi. the IL-10 is SEQ ID No: 5, the second antibody is an anti-BCMA monoclonal antibody, and Z is IL-28;
ccxxii. the IL-10 is SEQ ID No: 5, the second antibody is an anti-BCMA monoclonal antibody, and Z is IL-29;
ccxxiii. the IL-10 is SEQ ID No: 5, the second antibody is an anti-BCMA monoclonal antibody, and Z is IFN-α;
ccxxiv. the IL-10 is SEQ ID No: 5, the second antibody is an anti-BCMA monoclonal antibody, and Z is IL-21;
ccxxv. the IL-10 is SEQ ID No: 5, the second antibody is an anti-gpA33 monoclonal antibody, and Z is IL-2;
ccxxvi. the IL-10 is SEQ ID No: 5, the second antibody is an anti-gpA33 monoclonal antibody, and Z is IL-15;
ccxxvii. the IL-10 is SEQ ID No: 5, the second antibody is an anti-gpA33 monoclonal antibody, and Z is IL-7;
ccxxviii. the IL-10 is SEQ ID No: 5, the second antibody is an anti-gpA33 monoclonal antibody, and Z is IL-28;
ccxxix. the IL-10 is SEQ ID No: 5, the second antibody is an anti-gpA33 monoclonal antibody, and Z is IL-29;
ccxxx. the IL-10 is SEQ ID No: 5, the second antibody is an anti-gpA33 monoclonal antibody, and Z is IFN-α;
ccxxxi. the IL-10 is SEQ ID No: 5, the second antibody is an anti-gpA33 monoclonal antibody, and Z is IL-21;
ccxxxii. the IL-10 is SEQ ID No: 5, the second antibody is an anti-CD33 monoclonal antibody, and Z is IL-2;
ccxxxiii. the IL-10 is SEQ ID No: 5, the second antibody is an anti-CD33 monoclonal antibody, and Z is IL-15;
ccxxxiv. the IL-10 is SEQ ID No: 5, the second antibody is an anti-CD33monoclonal antibody, and Z is IL-7;
ccxxxv. the IL-10 is SEQ ID No: 5, the second antibody is an anti-CD33 monoclonal antibody, and Z is IL-28;
ccxxxvi. the IL-10 is SEQ ID No: 5, the second antibody is an anti-CD33 monoclonal antibody, and Z is IL-29;
ccxxxvii. the IL-10 is SEQ ID No: 5, the second antibody is an anti-CD33 monoclonal antibody, and Z is IFN-α;
ccxxxviii. the IL-10 is SEQ ID No: 5, the second antibody is an anti-CD33 monoclonal antibody, and Z is IL-21;
ccxxxix. the IL-10 is SEQ ID No: 5, the second antibody is an anti-DLL3 monoclonal antibody, and Z is IL-2;
ccxl. the IL-10 is SEQ ID No: 5, the second antibody is an anti-DLL3 monoclonal antibody, and Z is IL-15;
ccxli. the IL-10 is SEQ ID No: 5, the second antibody is an anti-DLL3 monoclonal antibody, and Z is IL-7;
ccxlii. the IL-10 is SEQ ID No: 5, the second antibody is an anti-DLL3 monoclonal antibody, and Z is IL-28;
ccxliii. the IL-10 is SEQ ID No: 5, the second antibody is an anti-DLL3 monoclonal antibody, and Z is IL-29;
ccxliv. the IL-10 is SEQ ID No: 5, the second antibody is an anti-DLL3 monoclonal antibody, and Z is IFN-α;
ccxlv. the IL-10 is SEQ ID No: 5, the second antibody is an anti-DLL3 monoclonal antibody, and Z is IL-21;
ccxlvi. the IL-10 is SEQ ID No: 5, the second antibody is an anti-MUC17 monoclonal antibody, and Z is IL-2;
ccxlvii. the IL-10 is SEQ ID No: 5, the second antibody is an anti-MUC17 monoclonal antibody, and Z is IL-15;
ccxlviii. the IL-10 is SEQ ID No: 5, the second antibody is an anti-MUC17 monoclonal antibody, and Z is IL-7;
ccxlix. the IL-10 is SEQ ID No: 5, the second antibody is an anti-MUC17 monoclonal antibody, and Z is IL-28;
ccl. the IL-10 is SEQ ID No: 5, the second antibody is an anti-MUC17 monoclonal antibody, and Z is IL-29;
ccli. the IL-10 is SEQ ID No: 5, the second antibody is an anti-MUC17 monoclonal antibody, and Z is IFN-α;
cclii. the IL-10 is SEQ ID No: 5, the second antibody is an anti-MUC17 monoclonal antibody, and Z is IL-21;
ccliii. the IL-10 is SEQ ID No: 5, the second antibody is an anti-CLDN18 monoclonal antibody, and Z is IL-2;
ccliv. the IL-10 is SEQ ID No: 5, the second antibody is an anti-CLDN18 monoclonal antibody, and Z is IL-15;
cclv. the IL-10 is SEQ ID No: 5, the second antibody is an anti-CLDN18 monoclonal antibody, and Z is IL-7;
cclvi. the IL-10 is SEQ ID No: 5, the second antibody is an anti-CLDN18 monoclonal antibody, and Z is IL-28;
cclvii. the IL-10 is SEQ ID No: 5, the second antibody is an anti-CLDN18 monoclonal antibody, and Z is IL-29;
cclviii. the IL-10 is SEQ ID No: 5, the second antibody is an anti-CLDN18 monoclonal antibody, and Z is IFN-α;
cclix. the IL-10 is SEQ ID No: 5, the second antibody is an anti-CLDN18 monoclonal antibody, and Z is IL-21;
cclx. the IL-10 is SEQ ID No: 5, the second antibody is an anti-GD2 monoclonal antibody, and Z is IL-2;
cclxi. the IL-10 is SEQ ID No: 5, the second antibody is an anti-GD2 monoclonal antibody, and Z is IL-15;
cclxii. the IL-10 is SEQ ID No: 5, the second antibody is an anti-GD2 monoclonal antibody, and Z is IL-7;
cclxiii. the IL-10 is SEQ ID No: 5, the second antibody is an anti-GD2 monoclonal antibody, and Z is IL-28;
cclxiv. the IL-10 is SEQ ID No: 5, the second antibody is an anti-GD2 monoclonal antibody, and Z is IL-29;
cclxv. the IL-10 is SEQ ID No: 5, the second antibody is an anti-GD2 monoclonal antibody, and Z is IFN-α;
cclxvi. the IL-10 is SEQ ID No: 5, the second antibody is an anti-GD2 monoclonal antibody, and Z is IL-21;
cclxvii. the IL-10 is SEQ ID No: 5, the second antibody is an anti-5T4 monoclonal antibody, and Z is IL-2;
cclxviii. the IL-10 is SEQ ID No: 5, the second antibody is an anti-5T4 monoclonal antibody, and Z is IL-15;
cclxix. the IL-10 is SEQ ID No: 5, the second antibody is an anti-5T4 monoclonal antibody, and Z is IL-7;
cclxx. the IL-10 is SEQ ID No: 5, the second antibody is an anti-5T4 monoclonal antibody, and Z is IL-28;
cclxxi. the IL-10 is SEQ ID No: 5, the second antibody is an anti-5T4 monoclonal antibody, and Z is IL-29;
cclxxii. the IL-10 is SEQ ID No: 5, the second antibody is an anti-5T4 monoclonal antibody, and Z is IFN-α;
cclxxiii. the IL-10 is SEQ ID No: 5, the second antibody is an anti-5T4 monoclonal antibody, and Z is IL-21;
cclxxiv. the IL-10 is SEQ ID No: 5, the second antibody is an anti-HER3 monoclonal antibody, and Z is IL-2;
cclxxv. the IL-10 is SEQ ID No: 5, the second antibody is an anti-HER3 monoclonal antibody, and Z is IL-15;
cclxxvi. the IL-10 is SEQ ID No: 5, the second antibody is an anti-HER3 monoclonal antibody, and Z is IL-7;
cclxxvii. the IL-10 is SEQ ID No: 5, the second antibody is an anti-HER3 monoclonal antibody, and Z is IL-28;
cclxxviii. the IL-10 is SEQ ID No: 5, the second antibody is an anti-HER3 monoclonal antibody, and Z is IL-29;
cclxxix. the IL-10 is SEQ ID No: 5, the second antibody is an anti-HER3 monoclonal antibody, and Z is IFN-α;
cclxxx. the IL-10 is SEQ ID No: 5, the second antibody is an anti-HER3 monoclonal antibody, and Z is IL-21;
cclxxxi. the IL-10 is SEQ ID No: 1, the second antibody is an anti-EGFR monoclonal antibody, and Z is IL-4;
cclxxxii. the IL-10 is SEQ ID No: 5, the second antibody is an anti-EGFR monoclonal antibody, and Z is IL-4;
cclxxxiii. the IL-10 is SEQ ID No: 1, the second antibody is an anti-HER2 monoclonal antibody, and Z is IL-4;
cclxxxiv. the IL-10 is SEQ ID No: 5, the second antibody is an anti-HER2 monoclonal antibody, and Z is IL-4;
cclxxxv. the IL-10 is SEQ ID No: 1, the second antibody is an anti-VEGFR2 monoclonal antibody, and Z is IL-4;
cclxxxvi. the IL-10 is SEQ ID No: 5, the second antibody is an anti-VEGFR2 monoclonal antibody, and Z is IL-4;
cclxxxvii. the IL-10 is SEQ ID No: 1, the second antibody is an anti-PDGFR monoclonal antibody, and Z is IL-4;
cclxxxviii. the IL-10 is SEQ ID No: 5, the second antibody is an anti-PDGFR monoclonal antibody, and Z is IL-4;
cclxxxix. the IL-10 is SEQ ID No: 1, the second antibody is an anti-GPC3 monoclonal antibody, and Z is IL-4;
ccxc. the IL-10 is SEQ ID No: 5, the second antibody is an anti-GPC3 monoclonal antibody, and Z is IL-4;
ccxci. the IL-10 is SEQ ID No: 1, the second antibody is an anti-PD-L1 monoclonal antibody, and Z is IL-4;
ccxcii. the IL-10 is SEQ ID No: 5, the second antibody is an anti-PD-L1 monoclonal antibody, and Z is IL-4;
ccxciii. the IL-10 is SEQ ID No: 1, the second antibody is an anti-CD19 monoclonal antibody, and Z is IL-4;
ccxciv. the IL-10 is SEQ ID No: 5, the second antibody is an anti-CD19 monoclonal antibody, and Z is IL-4;
ccxcv. the IL-10 is SEQ ID No: 1, the second antibody is an anti-CD20 monoclonal antibody, and Z is IL-4;
ccxcvi. the IL-10 is SEQ ID No: 5, the second antibody is an anti-CD20 monoclonal antibody, and Z is IL-4;
ccxcvii. the IL-10 is SEQ ID No: 1, the second antibody is an anti-CD22 monoclonal antibody, and Z is IL-4;
ccxcviii. the IL-10 is SEQ ID No: 5, the second antibody is an anti-CD22 monoclonal antibody, and Z is IL-4;
ccxcix. the IL-10 is SEQ ID No: 1, the second antibody is an anti-PSMA monoclonal antibody, and Z is IL-4;
ccc. the IL-10 is SEQ ID No: 5, the second antibody is an anti-PSMA monoclonal antibody, and Z is IL-4;
ccci. the IL-10 is SEQ ID No: 1, the second antibody is an anti-CEA monoclonal antibody, and Z is IL-4;
cccii. the IL-10 is SEQ ID No: 5, the second antibody is an anti-CEA monoclonal antibody, and Z is IL-4;
ccciii. the IL-10 is SEQ ID No: 1, the second antibody is an anti-BCMA monoclonal antibody, and Z is IL-4;
ccciv. the IL-10 is SEQ ID No: 5, the second antibody is an anti-BCMA monoclonal antibody, and Z is IL-4;
cccv. the IL-10 is SEQ ID No: 1, the second antibody is an anti-gpA33 monoclonal antibody, and Z is IL-4;
cccvi. the IL-10 is SEQ ID No: 5, the second antibody is an anti-gpA33 monoclonal antibody, and Z is IL-4;
cccvii. the IL-10 is SEQ ID No: 1, the second antibody is an anti-CD33 monoclonal antibody, and Z is IL-4;
cccviii. the IL-10 is SEQ ID No: 5, the second antibody is an anti-CD33 monoclonal antibody, and Z is IL-4;
cccix. the IL-10 is SEQ ID No: 1, the second antibody is an anti-DLL3 monoclonal antibody, and Z is IL-4;
cccx. the IL-10 is SEQ ID No: 5, the second antibody is an anti-DLL3 monoclonal antibody, and Z is IL-4;
cccxi. the IL-10 is SEQ ID No: 1, the second antibody is an anti-MUC17 monoclonal antibody, and Z is IL-4;
cccxii. the IL-10 is SEQ ID No: 5, the second antibody is an anti-MUC17 monoclonal antibody, and Z is IL-4;
cccxiii. the IL-10 is SEQ ID No: 1, the second antibody is an anti-CLDN18 monoclonal antibody, and Z is IL-4;
cccxiv. the IL-10 is SEQ ID No: 5, the second antibody is an anti-CLDN18 monoclonal antibody, and Z is IL-4;
cccxv. the IL-10 is SEQ ID No: 1, the second antibody is an anti-GD2 monoclonal antibody, and Z is IL-4;
cccxvi. the IL-10 is SEQ ID No: 5, the second antibody is an anti-GD2 monoclonal antibody, and Z is IL-4;
cccxvii. the IL-10 is SEQ ID No: 1, the second antibody is an anti-5T4 monoclonal antibody, and Z is IL-4;
cccxviii. the IL-10 is SEQ ID No: 5, the second antibody is an anti-5T4 monoclonal antibody, and Z is IL-4;
cccxix. the IL-10 is SEQ ID No: 1, the second antibody is an anti-HER3 monoclonal antibody, and Z is IL-4;
cccxx. the IL-10 is SEQ ID No: 5, the second antibody is an anti-HER3 monoclonal antibody, and Z is IL-4;
cccxxi. the IL-10 is SEQ ID No: 1, the second antibody is an anti-Ebola monoclonal antibody, and Z is IL-2;
cccxxii. the IL-10 is SEQ ID No: 1, the second antibody is an anti-Ebola monoclonal antibody, and Z is IL-15;
cccxxiii. the IL-10 is SEQ ID No: 1, the second antibody is an anti-Ebola monoclonal antibody, and Z is IL-7;
cccxxiv. the IL-10 is SEQ ID No: 1, the second antibody is an anti-Ebola monoclonal antibody, and Z is IL-28;
cccxxv. the IL-10 is SEQ ID No: 1, the second antibody is an anti-Ebola monoclonal antibody, and Z is IL-29;
cccxxvi. the IL-10 is SEQ ID No: 1, the second antibody is an anti-Ebola monoclonal antibody, and Z is IFN-α;
cccxxvii. the IL-10 is SEQ ID No: 1, the second antibody is an anti-Ebola monoclonal antibody, and Z is IL-21;
cccxxviii. the IL-10 is SEQ ID No: 5, the second antibody is an anti-Ebola monoclonal antibody, and Z is IL-2;
cccxxix. the IL-10 is SEQ ID No: 5, the second antibody is an anti-Ebola monoclonal antibody, and Z is IL-15;
cccxxx. the IL-10 is SEQ ID No: 5, the second antibody is an anti-Ebola monoclonal antibody, and Z is IL-7;
cccxxxi. the IL-10 is SEQ ID No: 5, the second antibody is an anti-Ebola monoclonal antibody, and Z is IL-28;
cccxxxii. the IL-10 is SEQ ID No: 5, the second antibody is an anti-Ebola monoclonal antibody, and Z is IL-29;
cccxxxiii. the IL-10 is SEQ ID No: 5, the second antibody is an anti-Ebola monoclonal antibody, and Z is IFN-α; or
cccxxxiv. the IL-10 is SEQ ID No: 5, the second antibody is an anti-Ebola monoclonal antibody, and Z is IL-21.
cccxxxv.

2. The method according to claim 1, wherein the diakine further comprises a scFv targeting domain that targets a receptor different from the BiTE or CAR-T.

3. The method according to claim 1, wherein the second anti-Ebola antibody of any one of cccxxi. to cccxxxiv. is engrafted.

4. The method according to claim 3, wherein the second anti-Ebola antibody is engrafted with three CDRs from a VH and three CDRs from a VL obtained from a monoclonal antibody selected from CD3, CD4, CD5, CD7, CD14, CD25, CD30, CD34, CD38, CD40, CD47, CD52, CD56, CD70, CD79B, CD117, CD123, CD138, CD147, C-type lectin-like molecule-1 (CLL01), PD-1, TIM3, BTLA, latent membrane protein 1 (LMP-1), signal lymphocytic activation molecule F7 (SLAMF7), NY-ESO-1, transmembrane activator and CAML interactor (TACI), CS-1, CXCR4, NKG2D, B7-H3, LAG3, CTLA4, VEGFR1, EpCAM, mesothelin (MSO), PSCA, PSA, MUC1, Lewis-Y, AXL, Claudin 18.2, ICAM-1, ICAM-2, ICAM-3, ICAM-4, ICAM-5, VCAM, FAPα, Trop2, EDB-FN, TGFβ, Trap, MAdCAM, β7 integrin subunit, α4β7 integrin, α4 integrin, SR-A1, SR-A3, SR-A4, SR-A5, SR-A6, SR-B, dSR-C1, SR-D1, SR-E1, SR-F1, SR-F2, SR-G, SR-H1, SR-H2, SR-I1, SR-J1, or HIV.

5. The method according to claim 1, wherein the CAR-T or BiTE therapy targets hematological or solid tumors.

6. The method according to claim 1, wherein the CAR-T or BiTE targets an antigenic determinant selected from TNFRSF17, IL3RA, SDC1, CD5, CD19, CD20, CD22, CD23, CD33, CD38, CD44, CD70, CD133, CD174, CD274, CD276, CEACAM6, GFRA1, ITGB6, MS4A1, TNFRSF8, NCAM1, ULBP1, ULBP2, IL1RAP, CEACAM5, MET, EGFR, EGFRvIII, ENPP1, FGFR4, EPCAM, EPHA2, ERBB2, GPC3, MSLN, Muc1, PDCD1, KDR, IL13RA2, FOLH1, FAP, CA9, FOLR1, L1CAM, ROR1, SLAMF7, GD2, PSCA, GPNMB, CSPG4, or TEM1.

7. The method according to claim 1, wherein the CAR-T therapy is idecabtagene vicleucel, lisocabtagene maraleucel, brexucabtagene autoleucel, tisagenlecleucel, or axicabtagene ciloleucel.
